(19) United States
(12) Patent Application Publication
(10) Pub. No.: US 2009/0226571 A1
(43) Pub. Date: Sep. 10, 2009

(54) Fermentative Production of Non-Volatile Microbial Metabolism Products in Solid Form

(75) Inventors: Stephan Freyer, Neustadt (DE); Markus Pompejus, Seoul (DE); Oskar Zelder, Speyer (DE); Markus Lohscheidt, Heidelberg (DE); Matthias Boy, Langen (DE); Edzard Scholten, Mannheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 11/991,515

(22) PCT Filed: Sep. 6, 2006
(86) PCT No.: PCT/EP2006/066057

§ 371 Date: Mar. 5, 2008

(30) Foreign Application Priority Data
Sep. 7, 2005 (DE) 10 2005 042 541.0

(51) Int. Cl. A23K 1/16; C12P 1/00; C12P 7/46; C12P 7/48; C12P 13/04; C12P 19/38; C12P 19/30; C12P 7/64; C12P 7/02; C12P 19/00; C12P 21/04; C12P 7/26; C12P 19/04; C12P 19/12; C12P 7/42; C12P 7/18; C07C 53/00; C07C 205/00; C07H 19/00; C07C 35/00; C07H 1/00; C07K 2/00; C07C 49/00; C08B 37/16; C13K 13/00; C07C 31/18; D06M 13/224; D06M 13/00; A23L 1/48; D21H 23/22

(52) U.S. Cl. 426/61; 435/41; 435/145; 435/144; 435/106; 435/87; 435/89; 435/134; 435/155; 435/72; 435/71.1; 435/148; 435/101; 435/100; 435/146; 435/158; 562/512; 562/553; 536/22.1; 554/1; 568/700; 536/1.11; 530/350; 568/303; 536/103; 536/123.13; 568/852; 252/8.81; 162/158

(57) ABSTRACT

The present invention relates to a method for the production of at least one nonvolatile microbial metabolite in solid form by sugar-based microbial fermentation, in which process a microorganism strain which produces the desired metabolites is grown using a sugar-containing liquid medium with a monosaccharide content of more than 20% by weight based on the total weight of the liquid medium, and the volatile constituents of the fermentation liquor are subsequently largely removed, the sugar-containing liquid medium being prepared by:
a1) milling selected starch feedstock from cereal grains; and
a2) liquefying the millbase in an aqueous liquid in the presence of at least one starch-liquefying enzyme, followed by saccharification using at least one saccharifying enzyme, where, for liquefaction purposes, at least a portion of the millbase is liquefied by continuous or batchwise addition to the aqueous liquid.

Furthermore, the invention relates to a solid formulation, of a nonvolatile microbial metabolite, obtainable by the method according to the invention; and to the use of such a solid formulation as additive or supplement for human or animal nutrition or for the treatment of textiles, leather, cellulose, paper or surfaces.

The present invention relates to the fermentative production of nonvolatile microbial metabolites in solid form by grinding, liquefying and saccharifying starch feedstocks selected among cereal grains and by using the resulting sugar-containing liquid medium for the fermentation.

Processes for the production of nonvolatile microbial metabolites such as, for example, amino acids, vitamins and carotenoids by microbial fermentation are generally known. Depending on the various process conditions, different carbon feedstocks are exploited for this purpose. They extend from pure sucrose via beet and sugarcane molasses, to what are known as high-test molasses (inverted sugarcane molasses) to glucose from starch hydrolyzates. Moreover, acetic acid and ethanol are mentioned as cosubstrates which can be employed on an industrial scale for the biotechnological production of L-lysine (Pfefferle et al., Biotechnological Manufacture of Lysine, Advances in Biochemical Engineering/Biotechnology, Vol. 79 (2003), 59-112).

Based on the abovementioned carbon feedstocks, various methods and procedures for the sugar-based, fermentative production of nonvolatile microbial metabolites are established. Taking L-lysine as an example, these are described for example by Pfefferle et al. (loc. cit.) with regard to strain development, process development and industrial production.

An important carbon feedstock for the microorganism-mediated fermentative production of nonvolatile microbial metabolites is starch. The latter must first be liquefied and saccharified in preceding reaction steps before it can be exploited as carbon feedstock in a fermentation. To this end, the starch is usually obtained in pre-purified form from a natural starch feedstock such as potatoes, cassava, cereals, for example wheat, maize (corn), barley, rye, triticale or rice, and subsequently enzymatically liquefied and saccharified, whereafter it is employed in the actual fermentation for producing the desired metabolites.

In addition to the use of such pre-purified starch feedstocks, the use of non-pretreated starch feedstocks for the preparation of carbon feedstocks for the fermentative production of nonvolatile microbial metabolites has also been described. Typically, the starch feedstocks are initially comminuted by grinding. The millbase is then subjected to liquefaction and saccharification. Since this millbase naturally comprises, besides starch, a series of nonstarchy constituents which adversely affect the fermentation, these constituents are usually removed prior to fermentation. The removal can be effected either directly after grinding (WO 02/277252; JP 2001-072701; JP 56-169594; CN 1218111), after liquefaction (WO 02/277252; CN 1173541) or subsequently to saccharification (CN 1266102; Beukema et al.: Production of fermentation syrups by enzymatic hydrolysis of potatoes; potato saccharification to give culture medium (Conference Abstract), Symp. Biotechnol. Res. Neth. (1983), 6; NL8302229). However, all variants involve the use of a substantially pure starch hydrolyzate in the fermentation.

More recent techniques deal in particular with improved methods which are intended to make possible, prior to the fermentation, a purification of, for example, liquefied and saccharified starch solutions (JP 57159500) and of fermentation media from renewable resources (EP 1205557).

In contrast, unprocessed starch feedstocks are known to be applied on a large scale in the fermentative production of bioethanol. The method of dry grinding, liquefying and saccharifying starch feedstocks, known as “dry milling”, is established industrially on a large scale. Descriptions of suitable processes can be found for example in “The Alcohol Textbook—A reference for the beverage, fuel and industrial alcohol industries”, Jaques et al. (Ed.), Nottingham Univ. Press 1995, ISBN 1-8977676-735, and in McAloon et al., “Determining the cost of producing ethanol from corn starch and lignocellulosic feedstocks”, NREL/TP-580-28893, National Renewable Energy Laboratory, October 2000.

In the dry-milling methods, intact cereal grains are ground finely in the first step, preferably maize, wheat, barley, sorghum and millet, and rye. In contrast to what is known as the “wet-milling” method, no additional liquid is added. The grinding into fine components has the purpose of making the starch which is present in the grains accessible to the effect of water and enzymes in the subsequent liquefaction and saccharification.

Since in the fermentative production of bioethanol the product of value is obtained by distillation, the use of starch feedstocks from the dry-milling process in non-pre-purified form does not constitute a particular problem. However, when using a dry-milling method for the production of nonvolatile microbial metabolites, the solids stream which is introduced into the fermentation via the sugar solution is problematic since it not only may have an adverse effect on the fermentation, but may also considerably complicate the subsequent workup.

Thus, the oxygen supply for the microorganisms employed is a limiting factor in many fermentations, in particular when the former have demanding oxygen requirements. In general, little is known about the effect of high solids concentrations on the transition of oxygen from the gas phase into the liquid phase, and thus on the oxygen transfer rate. On the other hand, it is known that a viscosity which increases with increasing solids concentration leads to a reduced oxygen transfer rate. If, moreover, surface-active substances are introduced into the fermentation medium together with the solids, they affect the tendency of the gas bubbles to coagulate. The resulting bubble size, in turn, has a substantial effect on oxygen transfer (Mersmann, A. et al.: Selection and Design of Aerobic Bioreactors, Chem. Eng. Technol. 13 (1990), 357-370).

As the result of the introduction of solids, a critical viscosity value of the media used can be reached as early as during the preparation of the starch-containing suspension since, for example, a suspension with more than 30% by weight of ground corn in water can no longer be mixed homogeneously (Industrial Enzymology, 2nd ed., T. Godfrey, S. West, 1996). This limits the glucose concentration in conventional procedures. As a rule, it is disadvantageous for process economical reasons to use solutions with a lower concentration since this results in a disproportionate dilution of the fermentation liquor. This causes the achievable final concentration of the target products to drop, which results in additional costs when these are isolated from the fermentation medium, and the space-time yield decreases, which, given an equal production quantity, leads to a higher volume requirement, i.e. higher investment costs.

Owing to these difficulties, prior-art variants of the dry-milling method are not suitable for providing starch feedstocks for the fermentative production of nonvolatile microbial metabolites and are therefore without particular economical importance. To date, attempts to apply the dry-milling concept and the advantages which exist in principle in connection with this method, to the industrial-scale production of nonvolatile microbial metabolites have only been described using cassava as starch feedstock.

Thus, while JP 2001/275693 describes a method for the fermentative production of amino acids in which peeled cassava tubers which have been ground in the dry state are employed as starch feedstock, it is necessary, to carry out the process, to adjust the particle size of the millbase to ≦150 μm. In the filtration step which is employed for this purpose, more than 10% by weight of the millbase employed, including non-starch-containing constituents, are removed before the starch comprised is liquefied/saccharified and subsequently fermented. A similar method is described in JP 2001/309751 for the production of an amino-acid-containing feed additive.

However, cassava should be relatively problem-free in relation to the dry-milling process in comparison with other starch feedstocks, in particular cereals or cereal grains. While the starch typically accounts for at least 80% by weight of the dry cassava root (Menezes et al., Fungal celluloses as an aid for the saccharification of Cassava, Biotechnology and Bioengineering, Vol. 20 (4), 1978, John Wiley and Sons, Inc., Table 1, page 558), the starch content (dry matter) in cereal is comparatively much lower, as a rule less than 70% by weight; for example it amounts to approximately 68% by weight in the case of corn and to approximately 65% by weight in the case of wheat (Jaques et al., The Alcohol Textbook, ibid.). Accordingly, the glucose solution obtained after liquefaction and saccharification comprises fewer contaminants, in particular fewer solids when dry-ground cassava is used. These contaminants and in particular the nonstarchy solids prove to be problematic when employing cereal grains as the starch feedstock since they account for a markedly greater portion in these starch feedstocks than in cassava. This is because the increased amount of contaminants substantially increases the viscosity of the reaction mixture.

Cassava starch, however, should be relatively easy to process. While it has a higher viscosity at the swelling temperature in comparison with corn starch, the viscosity, in contrast, drops more rapidly at increasing temperature in the case of cassava than in the case of corn starch for example (Menezes, T. J. B. de, Saccharification of Cassava for ethyl alcohol production, Process Biochemistry, 1978, page 24, right column). Moreover, the swelling and gelatinization temperatures of cassava starch are lower than those of starch from cereals such as corn, which is why it is more readily accessible to bacterial α-amylase than cereal starch (Menezes, T. J. B. de, loc. cit.).

Further advantages of cassava over cereal starch feedstocks are its low cellulose content and its low phytate content. Cellulose and hemicellulose can be converted into furfurals, in particular under acidic saccharification conditions (Jaques et al., The Alcohol Textbook, ibid.; Menezes, T. J. B. de, ibid.) which, in turn, may have an inhibitory effect on the microorganisms employed in the fermentation. Phytate likewise inhibits the microorganisms employed for the fermentation.

While it is thus possible, from a technical aspect, to process cassava as starch feedstock in a process which corresponds to the dry-milling process, such a cassava-based process is still complex, not optimized and therefore not widely used. Nothing has been reported to date about the use of cereals as starch feedstock in a method corresponding to the dry-milling process for the production of fine chemicals such as nonvolatile microbial metabolites.

WO 2005/116228 was the first to describe a sugar-based fermentative process for the microbial production of fine chemicals in which the starch feedstock employed is a millbase of cereal grains or other dry grains or seeds without removing the nonstarchy constituents prior to the fermentation. A substantial removal of the volatile constituents from the fermentation liquor, giving rise to a solid comprising the fermentation product, is not described.

It was an object of the present invention to provide an efficient process for the sugar-based fermentative production of nonvolatile microbial metabolites which permits the use of cereals, including corn, as starch feedstock. The process was to make possible a simple workup of the fermentation mixture, in particular by means of a drying process. Moreover, it was to be distinguished by easy handling of the media used and was to avoid, in particular, complicated pre-purification or main purification steps, such as, for example, the removal of solid nonstarchy constituents, prior to the fermentation.

In connection with work carried out by the applicant company, it has been found, surprisingly, that such a process can be carried out in an efficient manner despite the inherent increased solids content by liquefying, for the preparation of a sugar-containing liquid medium, a millbase obtained from cereal grains in an aqueous liquid in the presence of at least one starch-liquefying enzyme and subsequently saccharifying the mixture using at least one saccharifying enzyme, during which process, for liquefaction purposes, at least a portion of the millbase is added continuously or batchwise to the aqueous liquid in the course of the liquefaction.

The present invention thus relates to a process for the production of at least one nonvolatile microbial metabolite in solid form by sugar-based microbial fermentation, in which process a microorganism strain which produces the desired metabolite(s) is grown using a sugar-containing liquid medium with a monosaccharide content of more than 20% by weight based on the total weight of the liquid medium, and the volatile constituents of the fermentation liquor are subsequently largely removed, the sugar-containing liquid medium being prepared by:
a1) production of a millbase by milling a starch feedstock selected from cereal grains; and
a2) liquefying the millbase in an aqueous liquid in the presence of at least one starch-liquefying enzyme, followed by saccharification using at least one saccharifying enzyme,
   wherein, for liquefaction purposes, at least a portion of the millbase is added continuously or batchwise to the aqueous liquid in the course of the liquefaction.

Suitable as starch feedstock are, mainly, dry grains or seeds where the starch amounts to at least 40% by weight and preferably at least 50% by weight in the dried state. They are found in many of the cereal plants which are currently grown on a large scale, such as corn, wheat, oats, barley, rye, triticale, rice and various sorghum and millet species, for example sorgo and milo. The starch feedstock is preferably selected from corn, rye, triticale and wheat kernels. In principle, the process according to the invention can also be carried out with analogous starch feedstocks such as, for example, a mixture of various starch-containing analogous grains or seeds.

The sugars present in the sugar-containing liquid medium produced according to the invention are essentially monosaccharides such as hexoses and pentoses, for example glucose, fructose, mannose, galactose, sorbose, xylose, arabinose and ribose, in particular glucose. The amount of monosaccharides other than glucose can vary, depending on the starch feedstock used and the nonstarchy constituents present therein and may be affected by the conduct of the reaction, for example by the decomposition of cellulose constituents by addition of cellulases. The monosaccharides of the sugar-containing liquid medium advantageously comprise glucose in an amount of at least 60% by weight, preferably at least 70% by weight, and especially preferably at least 80% by weight, based on the total amount of sugars present in the sugar-containing liquid medium. Usually, the glucose amounts to in the range of from 75 to 99% by weight, in particular from 80 to 97% by weight and specifically from 85 to 95% by weight, based on the total amount of sugars present in the sugar-containing liquid medium.

The monosaccharide concentration, specifically the glucose concentration, in the liquid medium prepared in accordance with the invention is frequently at least 25% by weight, preferably at least 30% by weight, especially preferably at least 35% by weight, in particular at least 40% by weight, for example 25% to 55% by weight, in particular 30 to 52% by weight, especially preferably 35 to 50% by weight and specifically 40 to 48% by weight, based on the total weight of the liquid medium.

In accordance with the invention, the sugar-containing liquid medium with which the microorganism strain which produces the desired metabolites is cultured, comprises at least a portion, preferably at least 20% by weight, in particular at least 50% by weight, specifically at least 90% by weight and very specifically at least 99% by weight of the nonstarchy solid constituents which are present in the ground cereal grains, corresponding to the extraction rate. Based on the starchy constituents of the millbase (and thus on the amount of monosaccharide in the sugar-containing liquid medium), the nonstarchy solid constituents in the sugar-containing liquid medium preferably amount to at least 10% by weight and in particular to at least 25% by weight, for example to 25 to 75% by weight and specifically to 30 to 60% by weight.

To prepare the sugar-containing liquid medium, the starch feedstock in question is milled in step a1), with or without addition of liquid, for example water, preferably without addition of liquid. It is also possible to combine dry milling with a subsequent wet-milling step. Apparatuses which are typically employed for dry milling are hammer mills, rotor mills or roller mills; those which are suitable for wet milling are paddle mixers, agitated ball mills, circulation mills, disk mills, annular chamber mills, oscillatory mills or planetary mills. In principle, other mills are also suitable. The amount of liquid required for wet milling can be determined by the skilled worker in routine experiments. It is usually adjusted in such a way that the dry matter content is in the range of from 10 to 20% by weight.

Grinding brings about a particle size which is suitable for the subsequent process steps. In this context, it has proved advantageous when the millbase obtained in the milling step, in particular the dry milling step, in step a1) has flour particles, i.e. particulate constituents, with a particle size in the range of from 100 to 630 μm in an amount of from 30 to 100% by weight, preferably 40 to 95% by weight and especially preferably 50 to 90% by weight. Preferably, the millbase obtained comprises 50% by weight of flour particles with a particle size of more than 100 μm. As a rule, at least 95% by weight of the flour particles obtained have a particle size of less than 2 mm. In this context, the particle size is measured by means of screen analysis using a vibration analyzer. In principle, a small particle size is advantageous for obtaining a high product yield. However, an unduly small particle size may result in problems, in particular problems due to clump formation/agglomeration, when the millbase is slurried during liquefaction or processing, for example during drying of the solids after the fermentation step.

Usually, flours are characterized by the extraction rate or by the flour grade, whose correlation with one another is such that the characteristic of the flour grade increases with increasing extraction rate. The extraction rate corresponds to the amount by weight of the flour obtained based on 100 parts by weight of millbase applied. While, during the milling process, pure, ultrafine flour, for example from the interior of the cereal kernel, is initially obtained, the amount of crude fiber and husk content in the flour increases, while the proportion of starch decreases during further milling, i.e. with increasing extraction rate. The extraction rate is therefore also reflected in what is known as the flour grade, which is used as a figure for classifying flours, in particular cereal flours, and which is based on the ash content of the flour (known as ash scale). The flour grade or type number indicates the amount of ash (minerals) in mg which is left behind when 100 g of flour solids are incinerated. In the case of cereal flours, a higher type number means a higher extraction rate since the core of the cereal kernel comprises approximately 0.4% by weight of ash, while the husk comprises approximately 5% by weight of ash. In the case of a lower extraction rate, the cereal flours thus consist predominantly of the comminuted endosperm, i.e. the starch constituent of the cereal kernels; in the case of a higher extraction rate, the cereal flours also comprise the comminuted, protein-containing aleurone layer of the cereal grains; in the case of coarse mill, they also comprise the constituents of the protein-containing and fat-containing embryo and of the seed husks, which comprise raw fiber and ash. For the purposes of the invention, flours with a high extraction rate, or a high type number, are preferred in principle. If cereal is employed as starch feedstock, it is preferred that the intact kernels together with their husks are milled and processed, if appropriate after previously mechanically removing the germs and the husks.

To liquefy the starch present in the millbase, at least a portion of the millbase, preferably at least 40% by weight, in particular at least 50% by weight and very especially preferably at least 55% by weight, are introduced, in step a2), into the reactor in the course of the liquefaction step, but preferably before the saccharification step. Frequently, the added amount of millbase will not exceed 90% by weight, in particular 85% by weight and especially preferably 80% by weight, based on the total amount of millbase used. Typically, the portion of the millbase which is added in the course of the liquefaction is supplied to the reactor under conditions as prevail during the liquefaction step. The addition can be effected batchwise, i.e. portionwise, in several portions which preferably in each case do not amount to more than 20% by weight, especially preferably not more than 10% by weight, for example 1 to 20% by weight, in particular 2 to 10% by weight, of the total amount of the millbase to be liquefied, or else continuously. It is essential for the invention that only some of the millbase, preferably not more than 60% by weight, in particular not more than 50% by weight and especially preferably not more than 45% by weight of the millbase are present in the reactor at the beginning of the liquefaction process and that the remainder of the millbase is added during the liquefaction step.

The millbase may be added as a powder, i.e. without the addition of water, or as a suspension in an aqueous fluid, for example fresh water, recirculated process water, for example from the fermentation or the work-up.

The liquefaction can also be carried out continuously, for example in a multi-step reaction cascade.

In accordance with the invention, the liquefaction in step a2) is carried out in the presence of at least one starch-liquefying enzyme which is preferably selected from the α-amylases. Other enzymes which are active and stable under the reaction conditions and which liquefy stable starch can likewise be employed.

What follows relates to the use of α-amylases; however, it also applies generally to all starch-liquefying enzymes.

The α-amylase (or the starch-liquefying enzyme used) can be introduced first into the reaction vessel or added in the course of step a2). Preferably, a portion of the α-amylase required in step a2) is added at the beginning of step a2) or is first placed into the reactor. The total amount of α-amylase is usually in the range of from 0.002 to 3.0% by weight, preferably from 0.01 to 1.5% by weight and especially preferably from 0.02 to 0.5% by weight, based on the total amount of starch feedstock employed.

The liquefaction can be carried out above or below the gelling temperature. Preferably, the liquefaction in step a2) is carried out at least in part above the gelling temperature of the starch employed (known as the cooking process). As a rule, a temperature in the range of from 70 to 165° C., preferably from 80 to 125° C. and especially preferably from 85 to 115° C. is chosen, the temperature preferably being at least 5° C. and especially preferably at least 10° C. above the gelling temperature.

To achieve an optimal α-amylase activity, step a2) is preferably at least in part carried out at a pH in the weakly acidic range, preferably between 4.0 and 7.0, especially preferably between 5.0 and 6.5, the pH usually being adjusted before or at the beginning of step a2); preferably, this pH is checked during the liquefaction and, if appropriate, readjusted. The pH is preferably adjusted using dilute mineral acids such as H₂SO₄ or H₃PO₄, or dilute alkali hydroxide solutions such as aqueous sodium hydroxide solution (NaOH) or potassium hydroxide solution (KOH) or using alkaline-earth hydroxide solutions such as aqueous calcium hydroxide.

In a preferred embodiment, step a2) of the process according to the invention is carried out in such a way that a portion amounting to not more than 60% by weight, preferably not more than 50% by weight and especially preferably not more than 45% by weight, for example 10 to 60% by weight, in particular 15 to 50% by weight, and especially preferably 20 to 45% by weight, based on the total amount of millbase, is initially suspended in an aqueous liquid, for example fresh water, recirculated process water, for example from the fermentation or the processing stages, or in a mixture of these liquids, and the liquefaction is subsequently carried out. It is possible to preheat the liquid used for generating the suspension of the millbase to a moderately increased temperature, for example in the range of from 40 to 60° C. Preferably, the liquid applied for the preparation of the millbase suspension will not exceed 30° C. and will in particular have room temperature, i.e. 15 to 28° C.

Then, the at least one starch-liquefying enzyme, preferably an α-amylase, is added to this suspension. If an α-amylase is used, it is advantageous only to add a portion of the α-amylase, for example 10 to 70% by weight, in particular 20 to 65% by weight, based on all of the α-amylase employed in step a2). The amount of α-amylase added at this point in time depends on the activity of the α-amylase in question under the reaction conditions with regard to the starch feedstock used and is generally in the range of from 0.0004 to 2.0% by weight, preferably from 0.001 to 1.0% by weight and especially preferably from 0.02 to 0.3% by weight, based on the total amount of the starch feedstock employed. As an alternative, the α-amylase portion can be mixed with the liquid used before the suspension is made.

In this context, the α-amylase portion is preferably added to the suspension before heating to the temperature used for the liquefaction has started, in particular at room temperature or only moderately increased temperature, for example in the range of from 20 to 30° C.

Advantageously, the amounts of α-amylase and millbase will be selected in such a way that the viscosity during the saccharification process, in particular the gelling process is sufficiently reduced in order to make possible effective mixing of the suspension, for example by means of stirring. Preferably, the viscosity of the reaction mixture during gelling amounts to not more than 20 Pas, especially preferably not more than 10 Pas and very especially preferably not more than 5 Pas. As a rule, the viscosity is measured using a Haake viscometer type Roto Visko RV20 with M5 measuring system and MVDIN instrumentation at a temperature of 50° C. and a shear rate of 200 s⁻¹.

The suspension thus made is then heated, preferably at a temperature above the gelling temperature of the starch used. As a rule, a temperature in the range of from 70 to 165° C., preferably from 80 to 125° C. and especially preferably from 85 to 115° C. is chosen, the temperature preferably being at least 5° C. and especially preferably at least 10° C. above the gelling temperature. While monitoring the viscosity, further portions of the millbase, for example portionwise in amounts of in each case 2 to 20% by weight and in particular from 5 to 10% by weight, based on all of the millbase employed, are added gradually to the suspension of the millbase. It is preferred to add the portion of the millbase to be added in the course of the liquefaction step in at least 2, preferably at least 4 and especially preferably at least 6 fractions to the reaction mixture. As an alternative, the portion of the millbase which has not been employed for making the suspension can be added continuously during the liquefaction step. During the addition, the temperature should advantageously be kept above the gelling temperature of the starch. Preferably, the millbase is added in such a manner that the viscosity of the reaction mixture during the addition, or during the liquefaction process, amounts to no more than 20 Pas, especially preferably no more than 10 Pas and very especially preferably no more than 5 Pas.

After all of the millbase has been added, the reaction mixture is usually held for a certain period of time, for example 30 to 60 minutes or longer, if necessary, at the temperature set above the gelling temperature of the starch the starch constituents of the millbase being cooked. Then, the reaction mixture is, as a rule, cooled to a temperature slightly less above the gelling temperature, for example 75 to 90° C., before a further α-amylase portion, preferably the main portion, is added. Depending on the activity under the reaction conditions of the α-amylase used, the amount of α-amylase added at this point in time is preferably 0.002 to 2.0% by weight, especially preferably from 0.01 to 1.0% by weight and very especially preferably from 0.02 to 0.4% by weight, based on the total amount of the starch feedstock employed.

At these temperatures, the granular structure of the starch is destroyed (gelling), making possible the enzymatic degradation (liquefaction) of the latter. To fully degrade the starch into dextrins, the reaction mixture is held at the set temperature, or, if appropriate, heated further, until the detection of starch by means of iodine or, if appropriate, another test for detecting starch is negative or at least essentially negative. If appropriate, one or more further α-amylase portions, for example in the range of from 0.001 to 0.5% by weight and preferably from 0.002 to 0.2% by weight, based on the total amount of the starch feedstock employed, may now be added to the reaction mixture.

After the starch liquefaction has ended, the dextrins present in the liquid medium are saccharified, i.e. broken down into glucose, either continuously or batchwise, preferably continuously. The liquefied medium can be saccharified completely in a specific saccharification tank before being supplied to the fermentation step b). However, it has proved advantageous only to carry out a partial saccharification prior to the fermentation. For example, a procedure can be followed in which a portion of the dextrins present in the liquid medium, for example in the range of from 10 to 90% by weight and in particular in the range of from 20 to 80% by weight, based on the total weight of the dextrins (or of the original starch) is saccharified, and the resulting sugar-containing liquid medium is employed in the fermentation. A further saccharification can then be employed in the fermentation medium in situ. Moreover, the saccharification can be carried out directly in the fermentor (in situ), dispensing with a separate saccharification tank.

Advantages of the in-situ saccharification, i.e. of a saccharification which takes place in the fermentor, either in part or completely, are firstly a reduced outlay; secondly, a delayed liberation of the glucose allows, if appropriate, a higher glucose concentration to be provided in the batch without inhibition of or metabolic changes in the microorganisms employed being observed. In the case of E. coli, for example, an unduly high glucose concentration results in the formation of organic acids (acetate), while Saccharomyces cerevisae in such a case will, for example, switch to fermentation although a sufficient amount of oxygen is present in aerated fermentors (Crabtree effect). A delayed liberation of glucose can be adjusted by controlling the glucoamylase concentration. This allows the abovementioned effects to be suppressed, and more substrate can be initially introduced so that the dilution which is the result of the added feedstream, can be reduced.

In the case of separate saccharification, e.g. of saccharification in a saccharification tank, the liquefied starch solution is usually chilled or warmed to the temperature optimum of the saccharifying enzyme or slightly below, for example to 50 to 70° C., preferably 60 to 65° C., and subsequently treated with glucoamylase.

If the saccharification is carried out in the fermentor, the liquefied starch solution will, as a rule, be cooled to fermentation temperature, i.e. 32 to 37° C., before it is fed into the fermentor. In this case, the glucoamylase (or the at least one saccharifying enzyme) for the saccharification is added directly to the fermentation liquor. The saccharification of the liquefied starch in accordance with step a2) now takes place in parallel with the metabolization of the sugar by the microorganisms.

Prior to addition of the glucoamylase, the pH of the liquid medium is advantageously adjusted to a value in the optimal activity range of the glucoamylase employed, preferably in the range of from 3.5 to 6.0; especially preferably from 4.0 to 5.5 and very especially preferably from 4.0 to 5.0. However, it is also possible, in particular when carrying out the saccharification directly in the fermentor, to adjust the pH to a value outside the abovementioned ranges, for example in the range of from 6.0 to 8.0. This can be an overall advantage for example in the production of lysine, pantothenate and vitamin B₂, despite the limited activity of standard glucoamylases in this pH range, or may be required as the result of the fermentation conditions to be adjusted.

In a preferred embodiment, the saccharification is carried out in a specific saccharification tank. To this end, the liquefied starch solution is brought to and held at a temperature which is optimal for the enzyme, or slightly below, and the pH is adjusted in the above-described manner to a value which is optimal for the enzyme.

Usually, the glucoamylase is added to the dextrin-containing liquid medium in an amount of from 0.001 to 5.0% by weight, preferably from 0.005 to 3.0% by weight and especially preferably from 0.01 to 1.0% by weight, based on the total amount of the starch feedstock employed. After addition of the glucoamylase, the dextrin-containing suspension is preferably held for a period of, for example 2 to 72 hours or longer, if required, in particular 5 to 48 hours, at the set temperature, the dextrins being saccharified to give monosaccharides. The progress of the saccharification process can be monitored using methods known to the skilled worker, for example HPLC, enzyme assays or glucose test strips. The saccharification is complete when the monosaccharide concentration no longer rises substantially, or indeed drops.

In a preferred embodiment, the addition of the millbase in the presence of the at least one α-amylase and the at least one glucoamylase in step a2) is carried out in such a way that the viscosity of the liquid medium is not more than 20 Pas, preferably not more than 10 Pas and especially preferably not more than 5 Pas. To aid the control of the viscosity, it has proved advantageous to add at least 25% by weight, preferably at least 35% by weight and especially preferably at least 50% by weight of the total amount of the added millbase at a temperature above the gelatinization temperature of the starch present in the millbase. Moreover, controlling the viscosity can furthermore be influenced by adding the at least one starch-liquefying enzyme, preferably an α-amylase, and/or the at least one saccharifying enzyme, preferably a glucoamylase, portionwise themselves.

By practicing steps a1) and a2), it is possible to produce the sugar-containing liquid medium with a monosaccharide content, in particular a glucose content, of preferably more than 25% by weight, for example more than 30% by weight or more than 35% by weight, and especially preferably more than 40% by weight, for example >25 to 55% by weight, in particular >30 to 52% by weight, especially preferably >35 to 50% by weight and specifically >40 to 48% by weight, based on the total weight of the liquid medium. In such a case, the total solids content in the liquid medium will typically amount to 30 to 70% by weight, frequently 35 to 65% by weight, in particular 40 to 60% by weight. The monosaccharide, or glucose, concentration and the solids content depend in a manner known per se on the ratio of the millbase employed in the liquefaction and the amount of fluid, and on the starch content of the millbase.

Enzymes which can be used for liquefying the starch portion in the millbase are, in principle, all the α-amylases (enzyme class EC 3.2.1.1), in particular α-amylases obtained from Bacillus lichenformis or Bacillus staerothermophilus and specifically those which are used for liquefying materials obtained by dry-milling methods in connection with the production of bioethanol. The α-amylases which are suitable for the liquefaction are also commercially available, for example from Novozymes under the name Termamyl 120 L, type L; or from Genencor under the name Spezyme. A combination of different α-amylases may also be employed for the liquefaction.

Enzymes which can be used for saccharifying dextrins (i.e. oligosaccharides) in the liquefied starch solution are, in principle, all enzymes suitable for saccharifying dextrins, typically glucoamylases (enzyme class EC 3.2.1.3). In particular glucoamylases obtained from Aspergillus and specifically those which are used for saccharifying materials obtained by dry-milling methods in connection with the production of bioethanol are suitable. The enzymes which are suitable for the saccharification are also commercially available, for example from Novozymes under the name Dextrozyme GA; or from Genencor under the name Optidex. A combination of different saccharifying enzymes, e.g. different glucoamylases, may also be used.

To stabilize the enzymes employed, the concentration of Ca²⁺ ions may, if appropriate, be adjusted to an enzyme-specific optimum value, for example using CaCl₂ or Ca(OH)₂. Suitable concentration values can be determined by the skilled worker in routine experiments. If, for example, Termamyl is employed as α-amylase, it is advantageous to adjust the Ca²⁺ concentration to for example 50 to 100 ppm, preferably 60 to 80 ppm and especially preferably about 70 ppm in the liquid medium.

Since the entire starch feedstock is milled for the production of the sugar-containing liquid medium of a1), i.e the entire kernel, the nonstarchy solid constituents of the starch feedstock are also present. This frequently brings about the introduction of an amount of phytate from the grain, which amount is not to be overlooked. To avoid the inhibitory effect which thus results, it is advantageous to add, in step a2), at least one phytase to the liquid medium before subjecting the sugar-containing liquid medium to the fermentation step.

The phytase can be added before, during or after the liquefaction or the saccharification, if it is sufficiently stable to the respective high temperatures.

Any phytases can be employed as long as their activity is in each case not more than marginally affected under the reaction conditions. Phytases used preferably have a heat stability (T50)>50° C. and especially preferably >60° C.

The amount of phytase is usually from 1 to 10 000 units/kg starch feedstock and in particular 10 to 2000 units/kg starch feedstock.

To increase the overall sugar yield, or to obtain free amino acids, further enzymes, for example pullulanases, cellulases, hemicellulases, glucanases, xylanases, glucosidases or proteases, may additionally be added to the reaction mixture during the production of the sugar-containing liquid medium. The addition of these enzymes can have a positive effect on the viscosity, i.e. reduced viscosity (for example by cleaving longer-chain glucans and/or (arabino-)xylanes), and bring about the liberation of metabolizable glucosides and the liberation of (residual) starch. The use of proteases has analogous positive effects, it additionally being possible to liberate amino acids which act as growth factors for the fermentation.

In the process according to the invention the sugar-containing liquid medium is used for the fermentative production of a nonvolatile microbial metabolite. To this end, the sugar-containing liquid medium produced in steps a1) and a2) is subjected to a fermentation. The nonvolatile microbial metabolites are produced in the fermentation by the microorganisms.

As a rule, the fermentation process can be carried out in the generally known manner with which the skilled worker is familiar. The volumetric ratio between the fed sugar-containing liquid medium and the liquid medium which comprises the microorganisms and which has initially been introduced is generally in the range of from approximately 1:10 to 10:1, preferably in the range of from approximately 1:2 to 2:1, for example approximately 1:2 or approximately 2:1 and in particular approximately 1:1. The sugar content in the fermentation liquor can be controlled in particular via the feed rate of the sugar-containing liquid medium. As a rule, the feed rate will be adjusted in such a way that the monosaccharide content in the fermentation liquor is in the range of from ≧0% by weight to approximately 5% by weight; however, the fermentation can also be carried out at substantially higher monosaccharide contents in the fermentation liquor, for example approximately 5 to 20% by weight and in particular 10 to 20% by weight.

If the saccharification and the fermentation are carried out separately, the sugar-containing liquid medium obtained in step a) can, if appropriate, be sterilized before the fermentation, in which process any interfering microorganisms which may be present and which have been introduced for example together with the millbase (contaminants) are destroyed by a suitable method, typically by a thermal method. In the thermal method, the liquor is usually heated to temperatures of above 80° C. The destruction, or lysis, of the cells can take place immediately before the fermentation. To this end, all of the sugar-containing liquid medium is subjected to lysis or destruction. However, for the purposes of the present invention it has proved not to be necessary to carry out a sterilization step as described herein before the fermentation; rather, it has proved advantageous not to carry out such a sterilization step. Accordingly, a preferred embodiment of the invention relates to a process in which the liquid medium obtained in step a) (or steps a1) and a2), respectively) is fed directly to the fermentation, i.e. without previous sterilization or an at least partial in-situ saccharification is carried out.

The fermentation results in a liquid medium which, in addition to the desired, nonvolatile microbial metabolite and water, essentially comprises insoluble solids, e.g. the biomass generated during the fermentation, the nonmetabolized constituents of the saccharified starch solution and, in particular, the nonstarchy solid constituents of the starch feedstock such as, for example, fibers, and the constituents which are present in dissolved form in the fermentation liquor (soluble constituents), for example unutilized buffer and nutrient salts and unreacted monosaccharides (i.e. unutilized sugars). This liquid medium is hereinbelow also referred to as the fermentation liquor, the term fermentation liquor also comprising the (sugar-containing) liquid medium in which only a partial, or incomplete, fermentative conversion of the sugars present, i.e. a partial or incomplete microbial metabolization of the monosaccharides, has taken place.

In accordance with the invention, at least the volatile constituents of the fermentation medium are removed. In this manner, a solid is obtained which comprises the nonvolatile product of interest together with the unsoluble constituents of the fermentation liquor and, if appropriate, the components which are present in dissolved form in the fermentation liquor.

For the purposes of the present invention, nonvolatile microbial metabolites are understood as meaning compounds which, in general, cannot be removed from the fermentation liquor by distillation without undergoing decomposition.

As a rule, these compounds have a boiling point above the boiling point of water, frequently above 150° C. and in particular above 200° C. under normal pressure. As a rule, they take the form of compounds which are in the solid state under standard conditions (298 K, 101.3 kPa). However, it is also possible to employ the process according to the invention for the preparation of nonvolatile microbial metabolites which have a melting point below the boiling point of water and/or an oily consistency under atmospheric pressure. In this case, the maximum temperatures during processing, in particular during drying, will, as a rule, have to be controlled. These compounds can advantageously also be prepared by formulating them in pseudo-solid form on adsorbents.

Adsorbents which are suitable for this purpose are, for example, active charcoals, aluminas, silica gels, silicas, clay, soots, zeolites, inorganic alkali metal and alkaline earth metal salts such as the hydroxides, carbonates, silicates, sulfates and phosphates of sodium, potassium, magnesium and calcium, in particular magnesium and calcium salts, for example Mg(OH)₂, MgCO₃, MgSiO₄, CaSO₄, CaCO₃, alkaline earth metal oxides, for example MgO and CaO, other inorganic phosphates and sulfates, for example ZnSO₄, salts of organic acids, in particular their alkali metal and alkaline earth metal salts and specifically their sodium and potassium salts, for example the acetates, formates, hydrogen formates and citrates of sodium and potassium, and high-molecular-weight organic supports such as carbohydrates, for example sugars, optionally modified starches, cellulose, lignin, and generally the supports mentioned hereinbelow in the context of product formulation. As a rule, the abovementioned supports will contain no, or only small amounts, in particular only traces, of halogens such as chloride ions and of nitrates.

Examples of compounds which can be prepared advantageously in this manner by the process according to the invention are γ-linolenic acid, dihomo-γ-linolenic acid, arachidonic acid, eicosapentaenoic acid and docosahexaenoic acid, furthermore propionic acid, lactic acid, propanediol, butanol and acetone. Again, these compounds in pseudosolid formulation are understood as meaning nonvolatile microbial metabolites in solid form for the purposes of the present invention.

Hereinbelow, the term nonvolatile microbial metabolite comprises in particular organic mono-, di- and tricarboxylic acids which preferably have 3 to 10 carbon atoms and which, if appropriate, have one or more, for example 1, 2, 3 or 4, hydroxyl groups attached to them, for example tartaric acid, itaconic acid, succinic acid, propionic acid, lactic acid, 3-hydroxypropionic acid, fumaric acid, maleic acid, 2,5-furandicarboxylic acid, glutaric acid, levulic acid, gluconic acid, aconitic acid and diaminopimelic acid, citric acid; proteinogenic and nonproteinogenic amino acids, for example lysine, glutamate, methionine, phenyalalanine, aspartic acid, tryptophan and threonine; purine and pyrimidine bases; nucleosides and nucleotides, for example nicotinamide adenine dinucleotide (NAD) and adenosine-5′-monophosphate (AMP); lipids; saturated and unsaturated fatty acids having preferably 10 to 22 carbon atoms, for example γ-linolenic acid, dihomo-γ-linolenic acid, arachidonic acid, eicosapentaenoic acid and docosahexaenoic acid; diols having preferably 3 to 8 carbon atoms, for example propanediol and butanediol; higher-functionality alcohols having 3 or more, for example 3, 4, 5 or 6, OH groups, for example glycerol, sorbitol, mannitol, xylitol and arabinitol; longer-chain alcohols having at least 4 carbon atoms, for example 4 to 22 carbon atoms, for example butanol; carbohydrates, for example hyaluronic acid and trehalose; aromatic compounds, for example aromatic amines, vanillin and indigo; vitamins and provitamins, for example ascorbic acid, vitamin B₆, vitamin B₁₂ and riboflavin, cofactors and what are known as nutraceutics; proteins, for example enzymes such as amylases, pectinases, acid, hybrid or neutral cellulases, esterases such as lipases, pancreases, proteases, xylanases and oxidoreductases such as laccase, catalase and peroxidase, glucanases, phytases; carotenoids, for example lycopene, β-carotin, astaxanthin, zeaxanthin and canthaxanthin; ketones having preferably 3 to 10 carbon atoms and, if appropriate, 1 or more hydroxyl groups, for example acetone and acetoin; lactones, for example γ-butyrolactone, cyclodextrins, biopolymers, for example polyhydroxyacetate, polyesters, for example polylactide, polysaccharides, polyisoprenoids, polyamides; and precursors and derivatives of the abovementioned compounds. Other compounds which are suitable as nonvolatile microbial metabolites are described by Gutcho in Chemicals by Fermentation, Noyes Data Corporation (1973), ISBN: 0818805086.

The term “cofactor” comprises nonproteinaceous compounds which are required for the occurrence of a normal enzyme activity. These compounds can be organic or inorganic; preferably, the cofactor molecules of the invention are organic. Examples of such molecules are NAD and nicotinamide adenine dinucleotide phosphate (NADP); the precursor of these cofactors is niacin.

The term “nutraceutical” comprises food additives which promote health in plants and animals, in particular humans. Examples of such molecules are vitamins, antioxidants and certain lipids, for example polyunsaturated fatty acids.

The metabolites prepared are selected in particular among enzymes, amino acids, vitamins, disaccharides, aliphatic mono- and dicarboxylic acids having 3 to 10 carbon atoms, aliphatic hydroxycarboxylic acids having 3 to 10 carbon atoms, ketones having 3 to 10 carbon atoms, alkanols having 4 to 10 carbon atoms and alkanediols having 3 to 10 and in particular 3 to 8 carbon atoms.

The skilled worker will realize that the compounds produced by fermentation in accordance with the invention are obtained in each case in the enantiomeric form produced by the microorganisms employed (in the case where different enantiomers exist). Thus, for example, the respective L enantiomer will generally be obtained in the case of the amino acids.

The microorganisms employed in the fermentation depend in a manner known per se on the microbial metabolites in question, as specified in detail hereinbelow. They can be of natural origin or genetically modified. Examples of suitable microorganisms and fermentation processes are those given in Table A hereinbelow:

TABLE A

| Substance | Microorganism | Reference |
|---|---|---|
| Tartaric acid | Lactobacilli, (for | Rehm, H.-J.: Biotechnology, Weinheim, VCH, 1980 |
| | example | and 1993-1995; |
| | Lactobacillus | Gutcho, Chemicals by Fermentation, Noyes Data |
| | delbrueckii) | Corporation (1973), |
| Itaconic acid | Aspergillus terreus, | Jakubowska, in Smith and Pateman (Eds.), Genetics |
| | Aspergillus itaconicus | and Physiology of Aspergillus, London: Academic |
| | | Press 1977; Miall, in Rose (Ed.), Economic |
| | | Microbiology, Vol. 2, pp. 47-119, London: Academic |
| | | Press 1978; U.S. Pat. No. 3,044,941 (1962). |
| Succinic acid | Actinobacillus sp. | Int. J. Syst. Bacteriol. 26, 498-504 (1976); EP 249773 |
| | 130Z, | (1987), Inventors: Lemme and Datta; U.S. Pat. No. 5,504,004 |
| | Anaerobiospirillum | (1996), Inventors: Guettler, Jain and Soni; Arch. |
| | succiniproducens, | Microbiol. 167, 332-342 (1997); Guettler MV, Rumler D, |
| | Actinobacillus | Jain MK., Actinobacillus succinogenes sp. nov., a |
| | succinogenes, E. coli | novel succinic-acid-producing strain from the bovine |
| | | rumen. Int J Syst Bacteriol. 1999 Jan; 49 Pt 1: 207-16; |
| | | U.S. Pat. No. 5,723,322, U.S. Pat. No. 5,573,931, |
| | | U.S. Pat. No. 5,521,075, WO99/06532, |
| | | U.S. Pat. No. 5,869,301, U.S. Pat. No. 5,770,435 |
| Fumaric acid | Clostridium | Rhodes et al., Production of Fumaric Acid in 20-liter |
| | formicoaceticum, | Fermentors, Applied Microbiology, 1962, 10(1), 9-15; |
| | Rhizopus arrhizus | Dorn et al, Fermentation of Fumarate and L-Malate by |
| | | Clostridium formicoaceticum, Journal of Bacteriology, |
| | | 1978, 133(1), 26-32; NG et al., Production of |
| | | Tetrahydrofuran/1,4-butanediol by a combined |
| | | biological and chemical process, Biotechnology and |
| | | Bioengineering Symp. No. 17 (1986), pp. 355-363; WO |
| | | 90/00199 |
| Diaminopimelic | Corynebacterium | Rehm, H.-J.: Biotechnology, Weinheim, VCH, 1980 |
| acid | glutamicum | and 1993-1995; |
| | | Gutcho, Chemicals by Fermentation, Noyes Data |
| | | Corporation (1973), |
| Citric acid | Aspergillus niger, | Crit. Rev. Biotechnol. 3, 331-373 (1986); Food |
| | Aspergillus wentii | Biotechnol. 7, 221-234 (1993); 10, 13-27 (1996). |
| Aconitic acid | Aspergillus niger, | Crit. Rev. Biotechnol. 3, 331-373 (1986); Food |
| | Aspergillus wentii | Biotechnol. 7, 221-234 (1993); 10, 13-27 (1996).; |
| | | Rehm, H.-J.: Biotechnology, Weinheim, VCH, 1980 |
| | | and 1993-1995; |
| Malic acid | Aspergilli, for | U.S. Pat. No. 3,063,910; Battat et al., Optimization of L-Malic Acid |
| | example Aspergillus | Production by Aspergillus flavus in a Stirred |
| | flavus, A. niger, | Fermentor, Biotechnology and Bioengineering, Vol. 37 |
| | A. oryzae, | (1991), pp. 1108-1116 |
| | Corynebacterium | |
| Gluconic acid | Aspergilli, for | Gutcho, Chemicals by Fermentation, Noyes Data |
| | example A. niger | Corporation (1973), |
| Butyric acid | Clostridium (for | Rehm, H.-J.: Biotechnology, Weinheim, VCH, 1980 |
| | example Clostridium | and 1993-1995; |
| | acetobutlicum, | |
| | C. butyricum) | |
| Lysine | Corynebacterium | Ikeda, M.: Amino Acid Production Process (2003), Adv. |
| | glutamicum | Biochem. Engin/Biotechnol 79, 1-35. |
| Glutamate | Corynebacterium | Ikeda, M.: Amino Acid Production Process (2003), Adv. |
| | glutamicum | Biochem. Engin/Biotechnol 79, 1-35. |
| Methionine | Corynebacterium | Ikeda, M.: Amino Acid Production Process (2003), Adv. |
| | glutamicum | Biochem. Engin/Biotechnol 79, 1-35. |
| Phenyalalanine | Corynebacterium | Trends Biotechnol. 3, 64-68 (1985); J. Ferment. |
| | glutamicum, E. coli | Bioeng. 70, 253-260 (1990). |
| Threonine | E. coli | Ikeda, M.: Amino Acid Production Process (2003), Adv. |
| | | Biochem. Engin/Biotechnol 79, 1-35. |
| Aspartic acid | E. coli | Ikeda, M.: Amino Acid Production Process (2003), Adv. |
| | | Biochem. Engin/Biotechnol 79, 1-35 and references |
| | | cited therein, |
| | | Gutcho, Chemicals by Fermentation, Noyes Data |
| | | Corporation (1973) |
| Purine and | Bacillus subtilis | Rehm, H.-J.: Biotechnology, Weinheim, VCH, 1980 |
| pyrimidine bases | | and 1993-1995; |
| | | Gutcho, Chemicals by Fermentation, Noyes Data |
| | | Corporation (1973), |
| Nicotinamide | Bacillus subtilis | Rehm, H.-J.: Biotechnology, Weinheim, VCH, 1980 |
| adenine | | and 1993-1995; |
| dinucleotide | | Gutcho, Chemicals by Fermentation, Noyes Data |
| (NAD) | | Corporation (1973), |
| Adenosine-5′- | Bacillus subtilis | Rehm, H.-J.: Biotechnology, Weinheim, VCH, 1980 |
| monophosphate | | and 1993-1995; |
| (AMP) | | Gutcho, Chemicals by Fermentation, Noyes Data |
| | | Corporation (1973), |
| -Linolenic acid | Mucor, Mortiella, | Gill, I., Rao, V.: Polyunsaturated fatty acids, part 1: |
| | Aspergillus spp. | occurence, biological activities and applications (1997). |
| | | Trends in Biotechnology 15 (10), 401-409; Zhu, H.: |
| | | Utilization of Rice Brain by Pythium irregulare for Lipid |
| | | Production. Master Thesis Lousiana State University, |
| | | 31.10.2002 (URN etd-1111102-205855). |
| Dihomo- | Mortiella, | Gill, I., Rao, V.: Polyunsaturated fatty acids, part 1: |
| -linolenic acid | Conidiobolus, | occurence, biological activities and applications (1997). |
| | Saprolegnia spp. | Trends in Biotechnology 15 (10), 401-409; Zhu, H.: |
| | | Utilization of Rice Brain by Pythium irregulare for Lipid |
| | | Production. Master Thesis Lousiana State University, |
| | | 31.10.2002 (URN etd-1111102-205855). |
| Arachidonic acid | Mortiella, Phytium | Gill, I., Rao, V.: Polyunsaturated fatty acids, part 1: |
| | spp. | occurence, biological activities and applications (1997). |
| | | Trends in Biotechnology 15 (10), 401-409; Zhu, H.: |
| | | Utilization of Rice Brain by Pythium irregulare for Lipid |
| | | Production. Master Thesis Lousiana State University, |
| | | 31.10.2002 (URN etd-1111102-205855). |
| Eicosapentaenoic | Mortiella, Phytium | Gill, I., Rao, V.: Polyunsaturated fatty acids, part 1: |
| acid | spp., | occurence, biological activities and applications (1997). |
| | Rhodopseudomonas, | Trends in Biotechnology 15 (10), 401-409; Zhu, H.: |
| | Shewanella spp. | Utilization of Rice Brain by Pythium irregulare for Lipid |
| | | Production. Master Thesis Lousiana State University, |
| | | 31.10.2002 (URN etd-1111102-205855). |
| Docosahexaenoic | Thraustochytrium, | Gill, I., Rao, V.: Polyunsaturated fatty acids, part 1: |
| acid | Entomophthora spp., | occurence, biological activities and applications (1997). |
| | Rhodopseudomonas, | Trends in Biotechnology 15 (10), 401-409; Zhu, H.: |
| | Shewanella spp. | Utilization of Rice Brain by Pythium irregulare for Lipid |
| | | Production. Master Thesis Lousiana State University, |
| | | 31.10.2002 (URN etd-1111102-205855). |
| Butanediol | Enterobacter | Rehm, H.-J.: Biotechnology, Weinheim, VCH, 1980 |
| | aerogenes, Bacillus | and 1993-1995; |
| | subtilis, Klebsiella | Gutcho, Chemicals by Fermentation, Noyes Data |
| | oxytoca | Corporation (1973), |
| | | H. G. Schlegel, H. W. Jannasch, 1981; Afschar et al.: |
| | | Mikrobielle Produktion von 2,3-Butandiol, CIT 64 (6), |
| | | 2004, 570-571 |
| Glycerol | Yeast, | Gutcho, Chemicals by Fermentation, Noyes Data |
| | Saccharomyces | Corporation (1973), |
| | rouxii | |
| Mannitol | Aspergillus candidu, | Gutcho, Chemicals by Fermentation, Noyes Data |
| | Torulopsis | Corporation (1973), |
| | mannitofaciens | |
| Arabitol | Saccharomyces | Gutcho, Chemicals by Fermentation, Noyes Data |
| | rouxii, S. mellis, | Corporation (1973), |
| | Sclerotium | |
| | glucanicum, Pichia | |
| | ohmeri | |
| Xylitol | Saccharomyces | Gutcho, Chemicals by Fermentation, Noyes Data |
| | cerevisiae | Corporation (1973), |
| Hyaluronic acid | Streptococcus spp. | Rehm, H.-J.: Biotechnology, Weinheim, VCH, 1980 |
| | | and 1993-1995; |
| Trehalose | Brevibacterium, | JP 05099974, JP 06311891, FR 2671099, EP |
| | Corynebacterium, | 0555540, JP 3053791, Miyazaki, J.-I., Miyagawa, K.-I., |
| | Microbacterium, | Sugiyama, Y.: Trehalose Accumulation by |
| | Arthrobacter spp., | Basidiomycotinous Yeast, Filobasidium floriforme. |
| | Pleurotus genus, | Journal of Fermentation and Bioengineering 81, (1996) |
| | Filobasidium | 4, 315-319. |
| | floriforme | |
| Ascorbic acid | Gluconobacter | RÖMPP Online, Version 2.6, Georg Thieme Verlag KG |
| | melanogenes | |
| Vitamin B₁₂ | Propionibacterium | Chem. Ber. 1994, 923-927; RÖMPP Online, Version |
| | spp., Pseudomonas | 2.6, Georg Thieme Verlag KG |
| | denitrificans | |
| Riboflavin | Bacillus subtilis, | WO 01/011052, DE 19840709, WO 98/29539, |
| | Ashbya Gossypii | EP 1186664; Fujioka, K.: New biotechnology for |
| | | riboflavin (vitamin B2) and character of this riboflavin. |
| | | Fragrance Journal (2003), 31(3), 44-48. |
| Vitamin B₆ | Rhizobium tropici , R. meliloti | EP0765939 |
| Enzymes | Aspergilli (for | Rehm, H.-J.: Biotechnology, Weinheim, VCH, 1980 |
| | example Aspergillus | and 1993-1995; |
| | niger A. oryzae), | Gutcho, Chemicals by Fermentation, Noyes Data |
| | Trichoderma, E. coli, | Corporation (1973), |
| | Hanseluna or Pichia | |
| | (for example Pichia | |
| | pastorius), Bacillus | |
| | (for example Bacillus | |
| | licheniformis | |
| | B. subtilis) and many | |
| | others | |
| Zeaxanthin | Dunaliella salina | Jin et al (2003) Biotech.Bioeng. 81: 115-124 |
| Canthaxanthin | Brevibacterium | Nelis et al (1991) J Appl Bacteriol 70: 181-191 |
| Lycopene | Blakeslea trispora, | WO 03/056028, EP 01/201762, WO 01/12832, |
| | Candida utilis | WO 00/77234, |
| | | Miura et al (1998) Appl Environ Microbiol |
| | | 64: 1226-1229 |
| β-Carotene | Blakeslea trispora, | Kim S., Seo W., Park Y., Enhanced production of beta- |
| | Candida utilis | carotene from Blakeslea trispora with Span 20, |
| | | Biotechnology Letters, Vol 19, No 6, 1997, 561-562; |
| | | Mantouridou F., Roukas T.: Effect of the aeration rate |
| | | and agitation speed on beta-carotene production and |
| | | morphology of Blakeslea trispora in a stirred tank |
| | | reactor: mathematical modelling, Biochemical |
| | | Engineering Journal 10 (2002), 123-135; |
| | | WO 93/20183; WO 98/03480, Miura et al (1998) Appl |
| | | Environ Microbiol 64: 1226-1229 |
| Astaxanthin | Phaffia Rhodozyma; | US 00/5599711; US 90/00558; WO 91/02060, |
| | Candida utilis | Miura et al (1998) Appl Environ Microbiol |
| | | 64: 1226-1229 |
| Polyesters | Escherchia coli, | S. Y. Lee, Plastic Bacteria? Progress and Prospects |
| | Alcaligenes latus, and | for polyhydroxyalkanoate production in bacteria, |
| | many others | Tibtech, Vo. 14, (1996), pp. 431-438., Steinbüchel, |
| | | 2003; Steinbüchel (Ed.), Biopolymers, 1st ed., 2003, |
| | | Wiley-VCH, Weinheim and references cited therein |
| Polysaccharides | Leuconostoc | Rehm, H.-J.: Biotechnology, Weinheim, VCH, 1980 |
| | mesenteroides, L. dextranicum, | and 1993-1995; |
| | Xanthomonas | Gutcho, Chemicals by Fermentation, Noyes Data |
| | campestris, and | Corporation (1973), |
| | many others | |
| Polyisoprenoides | Lactarius sp., | Steinbüchel (Ed.), Biopolymers, 1st ed., 2003, |
| | Hygrophorus sp., | Wiley-VCH, |
| | Russula sp. | Weinheim and references cited therein |
| Polyamides | Actinobacillus sp. | Steinbüchel (Ed.), Biopolymers, 1st ed., 2003, |
| | 130Z, | Wiley-VCH, |
| | Anaerobiospirillum | Weinheim and references cited therein |
| | succiniproducens, | |
| | Actinobacillus | |
| | succino genes, E. coli | |
| Vanillin | Pseudomonas putida, | Priefert, H., Rabenhorst, J., Seinbüchel, A. |
| | Amycolatopsis sp. | Biotechnological production of vanillin. Appl. Microbiol. |
| | | Biotechnol. 56, 296-314 (2001) |
| Indigo | Escherichia coli JB | Berry, A., Dodge, T. C., Pepsin, M., Weyler, W.: |
| | 102 | Application of metabolic engineering to improve both |
| | | the production and use of biotech indigo. Journal of |
| | | Industrial Microbiology & Biotechnology 28 (2002), |
| | | 127-133. |
| Hydroxypropionic | Lactobacillus | RÖMPP Online, Version 2.6, Georg Thieme Verlag KG |
| acid | delbruckii, L. leichmannii | |
| | or. | |
| | Sporolactobacillus | |
| | inulinus | |
| Propionic acid | Propionibacterium, | Rehm, H.-J.: Biotechnology, Weinheim, VCH, 1980 |
| | e.g. P. arabinosum, | and 1993-1995; |
| | P. schermanii, P. freudenreichii | Gutcho, Chemicals by Fermentation, Noyes Data |
| | Clostridium | Corporation (1973), |
| | propionicum | |
| Lactic acid | Lactobacillus e.g. L. delbruckii | Rehm, H.-J.: Biotechnology, Weinheim, VCH, 1980 |
| | L. leichmannii | and 1993-1995 |
| Butanol | Clostridium (e.g. | Rehm, H.-J.: Biotechnology, Weinheim, VCH, 1980 |
| | Clostridium | and 1993-1995; |
| | acetobutlicum, C. propionicum) | Gutcho, Chemicals by Fermentation, Noyes Data |
| | | Corporation (1973), |
| Propanediol | E. coli | DE 3924423, U.S. Pat. No. 440,379, WO 9635799, U.S. Pat. No. 5,164,309 |
| Acetone | Clostridium (e.g. | Rehm, H.-J.: Biotechnology, Weinheim, VCH, 1980 |
| | Clostridium | and 1993-1995; |
| | acetobutlicum, C. propionicum) | Gutcho, Chemicals by Fermentation, Noyes Data |
| | | Corporation (1973), |
| Acetoin | Enterobacter | Lengeler, J. W., Drews, G., Schlegel, H. G.: Ed., Biology |
| | aerogenes, | of the Procaryotes, Thieme, Stuttgart (1999), p. 307; |
| | Clostridium | RÖMPP Online, Version 2.6, Georg Thieme Verlag KG |
| | acetobutylicum, | |
| | Lactococcus lactis | |

Preferred embodiments of the process according to the invention relate to the production of enzymes such as phytases, xylanases, glucanases; amino acids such as lysine, methionine, threonine; vitamins such as pantothenic acid and riboflavin, precursors and derivatives thereof, and the production of the abovementioned mono-, di- and tricarboxylic acids, in particular aliphatic mono- and dicarboxylic acids having 3 to 10 Carbon atoms such as propionic acid, fumaric acid and succinic acid, aliphatic hydroxycarboxylic acids having 3 to 10 Carbon atoms such as lactic acid; of the abovementioned longer-chain alkanols, in particular alkanols having 4 to 10 Carbon atoms such as butanol; of the abovementioned diols, in particular alkanediols having 3 to 10 and in particular 3 to 8 Carbon atoms such as propanediol; of the abovementioned ketones, in particular ketones having 3 to 10 Carbon atoms such as acetone; and of the abovementioned carbohydrates and in particular disaccharides such as trehalose.

In a preferred embodiment, the microorganisms employed in the fermentation are therefore selected from among natural or recombinant microorganisms which produce at least one of the following metabolites: enzymes such as phytase, xylanase, glucanase; amino acids such as lysine, threonine and methionine; vitamins such as pantothenic acid and riboflavin; precursors and/or derivatives thereof; disaccharides such as trehalose; aliphatic mono- and dicarboxylic acids having 3 to 10 Carbon atoms such as propionic acid, fumaric acid and succinic acid; aliphatic hydroxycarboxylic acids having 3 to 10 Carbon atoms such as lactic acid; ketones having 3 to 10 Carbon atoms such as acetone; alkanols having 4 to 10 Carbon atoms such as butanol; and alkanediols having 3 to 8 carbon atoms such as propanediol.

In particular, the microorganisms are selected from among the genera Corynebacterium, Bacillus, Ashbya, Escherichia, Aspergillus, Alcaligenes, Actinobacillus, Anaerobiospirillum, Lactobacillus, Propionibacterium, Rhizopus and Clostridium, in particular, among strains of Corynebacterium glutamicum, Bacillus subtilis, Ashbya gossypii, Escherichia coli, Aspergillus niger or Alcaligenes latus, Anaerobiospirillum succiniproducens, Actinobacillus succinogenes, Lactobacillus delbrückii, Lactobacillus leichmannii, Propionibacterium arabinosum, Propionibacterium schermanii, Propionibacterium freudenreichii, Clostridium propionicum, Clostridium formicoaceticum, Clostridium acetobutlicum, Rhizopus arrhizus and Rhizopus oryzae.

In a specific preferred embodiment, the metabolite produced by the microorganisms in the fermentation is lysine. To carry out the fermentation, analogous conditions and procedures as have been described for other carbon feedstocks, for example in Pfefferle et al., loc. cit. and U.S. Pat. No. 3,708,395, can be employed. In principle, both a continuous and a batchwise (batch or fed-batch) mode of operation are suitable, with the fed-batch mode being preferred.

In a further especially preferred embodiment, the metabolite produced by the microorganisms in the fermentation is methionine. To carry out the fermentation, analogous conditions and procedures as have been described for other carbon feedstocks, for example in WO 03/087386 and WO 03/100072, may be employed.

In a further especially preferred embodiment, the metabolite produced by the microorganisms in the fermentation is pantothenic acid. To carry out the fermentation, analogous conditions and procedures as have been described for other carbon feedstocks, for example in WO 01/021772, may be employed.

In a further especially preferred embodiment, the metabolite produced by the microorganisms in the fermentation is riboflavin. To carry out the fermentation, analogous conditions and procedures as have been described for other carbon feedstocks, for example in WO 01/011052, DE 19840709, WO 98/29539, EP 1186664 and Fujioka, K: New biotechnology for riboflavin (vitamin B2) and character of this riboflavin. Fragrance Journal (2003), 31(3), 4448, may be employed.

In a further especially preferred embodiment, the metabolite produced by the microorganisms in the fermentation is fumaric acid. To carry out the fermentation, analogous conditions and procedures as have been described for other carbon feedstocks, for example in Rhodes et al, Production of Fumaric Acid in 20-L Fermentors, Applied Microbiology, 1962, 10 (1), 9-15, may be employed.

In a further especially preferred embodiment, the metabolite produced by the microorganisms in the fermentation is a phytase. To carry out the fermentation, analogous conditions and procedures may be employed as have been described for other carbon feedstocks, for example in WO 98/55599.

Before the fermentation liquor is subjected to further processing, a sterilization step is preferably carried out. The sterilization step can be performed thermally, chemically or mechanically, or by a combination of these measures. Thermal sterilization can be effected in the above-described manner. For the chemical sterilization, the fermentation liquor will, as a rule, be treated with acids or bases in such a manner that the destruction of the microorganisms results. Mechanical sterilization is, as a rule, performed by introducing shear forces. Such methods are known to the skilled worker.

The process according to the invention advantageously comprises the following three successive process steps a), b) and c):
a) preparation of the sugar-containing liquid medium with a monosaccharide content of more than 20% by weight as described in steps a1) and a2), where the sugar-containing liquid medium also comprises nonstarchy solid constituents of the starch feedstock;
b) use of the sugar-containing liquid medium in a fermentation in order to produce the nonvolatile metabolite(s) and
c) obtaining the nonvolatile metabolite(s) in solid form together with at least part of the nonstarchy solid constituents of the starch feedstock from the fermentation liquor by removing at least some of the volatile constituents of the fermentation liquor.

The sugar-containing liquid medium obtained in step a) in which the microorganism strain producing the desired metabolites is cultured in step b) comprises at least some or all, but as a rule at least 90% by weight and specifically approximately 100% by weight of the nonstarchy solid constituents present in the milled cereal kernels, depending on the extraction rate. Based on the starchy constituents of the millbase, the amount of the nonstarchy solid constituents in the sugar-containing liquid medium is preferably at least 10% by weight and in particular at least 25% by weight, for example from 25 to 75% by weight and specifically from 30 to 60% by weight. These nonstarchy solid constituents are supplied together with the sugar-containing liquid medium to the fermentation as described in step b) and are thus also present in the resulting metabolite-comprising fermentation liquor.

If desired, a portion, for example 5 to 80% by weight and in particular 30 to 70% by weight, of the nonstarchy solid, i.e. insoluble constituents can be separated from the fermentation liquor. Such a separation is typically effected by usual methods of solid-liquid separation, for example by means of centrifugation or filtration. If appropriate, such a preliminary separation will be carried out in order to remove coarser solids particles which comprise no, or only small amounts of, nonvolatile microbial metabolite. This primary filtration can be carried out using conventional methods which are known to the skilled worker, for example using coarse sieves, nets, perforated sheets or the like. If appropriate, coarse solids particles may also be separated off in a centrifugal-force separator. The equipment employed here, such as decanter, centrifuges, sedicanter and separators, are also known to the skilled worker. Preferably, however, no more than 30% by weight, in particular no more than 5% by weight, of the insoluble constituents of the fermentation liquor will be removed before the volatile constituents are removed.

Preferably, the at least one nonvolatile metabolite in solid form is essentially obtained from the fermentation liquor without previously separating off solid constituents, together with the totality of all solid constituents.

In accordance with the invention, the fermentation is followed by the substantial removal of the volatile constituents of the fermentation liquor, if appropriate after previously having separated off a portion of the solid nonstarchy constituents. Substantial means that, after the volatile constituents have been removed, a solid or at least semi-solid residue remains which, if appropriate, can be converted into a solid product by addition of solid substances. As a rule, this means that the volatile constituents are removed down to a residual moisture content of not more than 20% by weight, frequently not more than 15% by weight and in particular not more than 10% by weight. As a rule, the volatile constituents of the fermentation liquor will be removed from the fermentation liquor down to a residual moisture content of advantageously in the range of from 0.2 to 20% by weight, preferably 1 to 15% by weight, especially preferably 2 to 10% by weight and very especially preferably 5 to 10% by weight, based on the total weight of the solid constituents determined after drying. The residual moisture content can be determined by conventional processes which are known to the skilled worker, for example by means of thermal gravimetry (Hemminger et al., Methoden der thermischen Analyse, Springer Verlag, Berlin, Heidelberg, 1989).

To remove the volatile constituents of the fermentation liquor, it is possible to proceed, in accordance with a first embodiment, in such a manner that essentially only the volatile constituents of the fermentation liquor are removed, for example by evaporation.

In accordance with a second embodiment, the liquid components of the fermentation liquor which, in addition to the volatile constituents, also comprises, as a rule, dissolved nonvolatile constituents, are removed from the undissolved constituents, i.e. the desired metabolite and biomass and the nonstarchy solid constituents of the starch source. The liquid components are then removed by usual methods of solid-liquid separation such as filtration, centrifugation and the like.

These methods of the first and second embodiment may also be employed in combination. For example, it is possible initially to separate some or the majority of the liquid components of the fermentation liquor from the undissolved components and the residual volatile components can be removed from the separated undissolved components of the fermentation liquor by evaporation. Furthermore, it is possible to remove most or all of the volatile constituents from the separated liquid component of the fermentation liquor by evaporation and to process it. Also, it is possible to combine the residue which is obtained by evaporation of the volatile constituents from the separated liquid constituents with the solids obtained after separation of the liquid constituents, which may be especially advantageous from the process engineering angle.

Obtaining the nonvolatile metabolite(s) in solid form from the fermentation liquor in step c) can be accomplished in one, two or more steps, if appropriate after a previous preliminary separation, in particular in a one- or two-step procedure. As a rule, at least one, in particular the final, step for obtaining the metabolite in solid form will comprise a drying step.

In the case of the one-step procedure, the volatile constituents of the fermentation liquor will be removed, if appropriate after the abovementioned preliminary separation, until the desired residual moisture content is reached.

In the case of the two- or multi-step procedure, the fermentation liquor will first be concentrated, if appropriate after the abovementioned preliminary separation, for example by means of (micro-, ultra-) filtration or thermally by evaporating some of the volatile constituents. The amount of the volatile constituents which are removed in this step is, as a rule, from 10 to 80% by weight and in particular from 20 to 70% by weight, based on the total weight of the volatile constituents of the fermentation liquor. The remaining volatile constituents of the fermentation liquor are removed in one or more subsequent steps until the desired residual moisture content is reached.

In accordance with the invention, the volatile constituents of the liquid medium are essentially removed without previous depletion or indeed isolation of the product of value. As a consequence, when removing the volatile constituents of the fermentation liquor, the nonvolatile metabolite is essentially not removed together with the volatile constituents of the liquid medium, but remains with at least some, usually with most and in particular with all of the remaining solid constituents from the fermentation liquor in the residue thus obtained. However, some, preferably small, amounts of the desired nonvolatile microbial metabolite, as a rule not more than 20% by weight, for example from 0.1 to 20% by weight, preferably not more than 10, in particular not more than 5% by weight, especially preferably not more than 2.5% by weight and very especially preferably not more than 1% by weight, based on the total dry weight of the metabolite, can be removed in accordance with the invention together with the volatile constituents of the fermentation liquor as these are removed. In a very especially preferred embodiment, at least 90% by weight, in particular at least 95% by weight, specifically 99% by weight and very specifically approximately 100% by weight of the desired nonvolatile microbial metabolite, in each case based on the total dry weight of the metabolite, remains as solid in mixture with the portion, or all, of the solid constituents of the fermentation medium after the volatile constituents have been removed.

This gives a solid or semisolid, for example pasty, residue which comprises the nonvolatile metabolite and the nonvolatile, as a rule solid nonstarchy, constituents of the starch feedstock or at least large portions thereof, frequently at least 90% by weight or all of the solid nonstarchy constituents.

The properties of the dry metabolite, which is present together with the solid constituents of the fermentation, can be formulated in a manner known per se specifically with regard to a variety of parameters such as active substance content, particle size, particle shape, tendency to dust, hygroscopicity, stability, in particular storage stability, color, odor, flowing behavior, tendency to agglomerate, electrostatic charge, sensitivity to light and high temperatures, mechanical stability and redispersibility, by addition of formulation auxiliaries such as carrier and coating materials, binders and other additives.

The formulation auxiliaries which are conventionally used include, for example, binders, carriers, powdering/flow adjuvants, furthermore color pigments, biocides, dispersants, antifoams, viscosity regulators, acids, alkalis, antioxidants, enzyme stabilizers, enzyme inhibitors, adsorbates, fats, fatty acids, oils or mixtures of these. Such formulation auxiliaries are advantageously employed as drying aids in particular when using formulation and drying methods such as spray drying, fluidized-bed drying and freeze-drying.

Examples of binders are carbohydrates, in particular sugars such as mono-, di-, oligo- and polysaccharides, for example dextrins, trehalose, glucose, glucose syrup, maltose, sucrose, fructose and lactose; colloidal substances such as animal proteins, for example gelatin, casein, in particular sodium caseinate, plant proteins, for example soya protein, pea protein, bean protein, lupin, zein, wheat protein, maize protein and rice protein, synthetic polymers, for example polyethylene glycol, polyvinyl alcohol and in particular the Kollidon brands by BASF, optionally modified biopolymers, for example lignin, chitin, chitosan, polylactid and modified starches, for example octenyl succinate anhydride (OSA); gums, for example acacia gum; cellulose derivatives, for example methylcellulose, ethylcellulose, (hydroxyethyl)methylcellulose (HEMC), (hydroxypropyl)methylcellulose (HPMC), carboxymethylcellulose (CMC); meals, for example ground maize, wheat, rye, barley and rice.

Examples of carriers are carbohydrates, in particular the sugars mentioned hereinabove as binders, and starches, for example from maize, rice, potato, wheat and cassava; modified starches, for example octenyl succinate anhydride; cellulose and microcrystalline cellulose; inorganic minerals or loam, for example clay, coal, kieselguhr, silica, tallow and kaolin; coarse meals, for example coarse wheatmeal, bran, for example wheat bran, the meals mentioned hereinabove as binders; salts such as metal salts, in particular alkali metal and alkaline earth metal salts of organic acids, for example Mg citrate, Mg acetate, Mg formate, Mg hydrogenformate, Ca citrate, Ca acetate, Ca formate, Ca hydrogenformate, Zn citrate, Zn acetate, Zn formate, Zn hydrogenformate, Na citrate, Na acetate, Na formate, Na hydrogenformate, K citrate, K acetate, K formate, K hydrogenformate, inorganic salts, for example Mg sulfates, Mg carbonates, Mg silicates or Mg phosphates, Ca sulfates, Ca carbonates, Ca silicates or Ca phosphates, Zn sulfates, Zn carbonates, Zn silicates or Zn phosphates, Na sulfates, Na carbonates, Na silicates or Na phosphates, K sulfates, K carbonates, K silicates or K phosphates, alkaline earth metal oxides such as CaO and MgO; inorganic buffering agents such as alkali metal hydrogen phosphates, in particular sodium and potassium hydrogen phosphates, for example K₂HPO₄, KH₂PO₄ and Na₂HPO₄; and generally the adsorbents mentioned in connection with the preparation according to the invention of metabolites with a low melting point or oily consistency.

Examples of powdering adjuvants or flow adjuvants are kieselguhr, silica, for example the Sipernat brands by Degussa; clay, coal, tallow and kaolin; the starches, modified starches, inorganic salts, salts of organic acids and buffering agents which have been mentioned above as carriers; cellulose and microcrystalline cellulose.

As regards other additives, the following may be mentioned by way of example: color pigments such as TiO₂, carotenoids and their derivatives, vitamin B₂, capsanthin, lutein, kryptoxanthin, canthaxanthin, astaxanthin, tartrazine, Sunset Yellow FCF, indigotin, vegetable charcoal, bixin, iron oxide; biocides such as sodium benzoate, sorbic acid, alkali metal sorbates and alkaline earth metal sorbates such as sodium sorbate, potassium sorbate and calcium sorbate, ethyl 4-hydroxybenzoate, alkali metal bisulfites such as sodium bisulfite and sodium metabisulfite, formic acid, formates and in particular alkali metal formates such as sodium formate, formaldehyde, sodium nitrate, acetates and in particular alkali/alkaline earth metal acetates such as sodium acetate and potassium acetate, acetic acid, lactic acid, propionic acid, dispersants and viscosity regulators such as alginates, lecithin, 1,2-propanediol, agar, carrageenan, gum arabic, guar gum, xanthan gum, gellan gum, cassia gum, sorbitol, polyethylene glycol, glycerol, pectin, modified starches, modified celluloses (for example methylcellulose, HPMC, ethylcellulose, carboxymethylcellulose), microcrystalline cellulose, mono- and diglycerides, sucrose esters; antifoam agents such as vinyl-functional silicone oils, for example SILOFOAM™SC 155 from Wacker Chemie, and fatty alcohol alkoxylates, for example Plurafac® from BASF AG; inorganic acids such as phosphoric acids, nitric acid, hydrochloric acid, sulfuric acid; organic acids such as saturated and unsaturated mono- and dicarboxylic acids, for example formic acid, acetic acid, propionic acid, butyric acid, valeric acid, palmitic acid, stearic acid, oxalic acid, malonic acid, succinic acid, glutaric acid, adipic acid, pimelic acid, maleic acid and fumaric acid; bases such as alkali metal hydroxides, for example NaOH and KOH; antioxidants such as vitamin C, 3-tert-butyl-4-hydroxyanisole (BHA), 3,5-di-tert-4-hydroxytoluene (BHT), 6-ethoxy-1,2-dihydroxy-2,2,4-trimenthylquinoline (ethoxyquin); enzyme stabilizers such as calcium salts, zinc salts such as zinc sulfate, magnesium salts such as magnesium sulfate, amino acids; enzyme inhibitors such as pepstatin A or guanidine*HCl; adsorbates such as silica, silicon oxide, sugars or salts; fats such as glycerides, for example mono-, di- and triglycerides; fatty acids such as stearic acid; oils such as sunflower oil, corn oil, soya oil and palm oil.

The amount of the abovementioned additives and, if appropriate, further additives such as coating materials can vary greatly, depending on the specific requirements of the metabolite in question and on the properties of the additives employed and can be for example in the range of from 0.1 to 80% by weight and in particular in the range of from 1 to 30% by weight, in each case based on the total weight of the product or substance mixture in its finished formulated form.

The addition of formulation auxiliaries can be effected before, during or after working up the fermentation liquor (also referred to as product formulation or solids design), in particular during drying. An addition of formulation auxiliaries before working up the fermentation liquor or the metabolite can be advantageous in particular for improving the processability of the substances or products to be worked up. The formulation auxiliaries can be added either to the metabolite obtained in solid form or else to a solution or suspension comprising the metabolite, for example directly to the fermentation liquor after the fermentation has been completed or to a solution or suspension obtained during workup and before the final drying step.

Thus, for example, the auxiliaries can be admixed with a suspension of the microbial metabolite; such a suspension can also be applied to a carrier material, for example by spraying on or mixing in. The addition of formulation auxiliaries during drying can be of importance for example when a solution or suspension comprising the metabolite is being sprayed. An addition of formulation auxiliaries is effected in particular after drying, for example when applying coatings/coating layers to dried particles. Further adjuvants can be added to the product both after drying and after an optional coating step.

Removing the volatile constituents from the fermentation liquor is effected in a manner known per se by customary methods for separating solid phases from liquid phases, including filtration methods and methods of evaporating volatile constituents of the liquid phases. Such methods, which may also encompass steps for roughly cleaning the products of value and formulation steps, are described, for example in Belter, P. A, Bioseparations: Downstream Processing for Biotechnology, John Wiley & Sons (1988), and Ullmann's Encyclopedia of Industrial Chemistry, 5th ed. on CD-ROM, Wiley-VCH. Methods, equipment, auxiliaries and general or specific embodiments which are known to the skilled worker which can be employed within the scope of product formulation or work-up after the fermentation has ended are furthermore described in EP 1038 527, EP 0648 076, EP 835613, EP 0219 276, EP 0394 022, EP 0547 422, EP 1088 486, WO 98/55599, EP 0758 018 and WO 92/12645.

In a first, preferred embodiment of the separation of the volatile constituents from the product of value and the nonstarchy solid constituents of the fermentation liquor, the nonvolatile microbial metabolite, if present in dissolved form in the liquid phase, will be converted from the liquid phase into the solid phase, for example by crystallization or precipitation. Thereafter, the nonvolatile solid constituents, including the metabolite, from the liquid constituents are separated by means of a customary method of solid-liquid separation, for example by means of centrifugation, decanting or filtration. Oily metabolites may also be separated off in a similar manner, the oily fermentation products in question being converted into a solid form by addition of adsorbents, for example silica, silica gels, loam, clay and active charcoal.

The precipitation of the microbial metabolites may be effected in a conventional manner (J. W. Mullin: Crystallization, 3rd ed., Butterworth-Heinemann, Oxford 1993). The precipitation can be initiated for example by addition of a further solvent, addition of salts and the variation of the temperature. The resulting precipitate can be separated from the liquor, together with the other solid constituents, by the herein-described conventional methods for separating solids.

The crystallization of microbial metabolites can likewise be accomplished in the customary manner. Customary crystallization processes are described, for example, in Janeic, S. J., Grootscholten, P. A., Industrial Crystallization, New York, Academic, 1984; A. W. Bamforth: Industrial Crystallization, Leonard Hill, London 1965; G. Matz: Kristallisation, 2nd edition, Springer Verlag, Berlin 1969; J. N\grave{y}vlt: Industrial Crystallization—State of the Art. VCH Verlagsges, Weinheim 1982; S. J. Jancic', P. A. M. Grootscholten: Industrial Crystallization, Reidel, Dordecht 1984; O. Söhnel, J. Garside: Precipitation, Butterworth-Heinemann, Oxford, 1992; A. S. Myerson (Ed.): Handbook of Industrial Crystallization, Butterworth-Heineman, Boston 1993; J. W. Mullin: Crystallization, 3rd edition, Butterworth-Heinemann, Oxford 1993; A. Mersmann (Ed.): Crystallization Technology Handbook, Marcel Dekker, New York 1995. A crystallization can be initiated for example by cooling, evaporation, crystallization in vacuo (adiabatic cooling), reaction crystallization or salting out. The crystallization can be performed for example in stirred and unstirred vessels, by the direct contact method, in evaporative crystallizers (R. K. Multer, Chem. Eng. (N.Y.) 89 (1982) March, 87-89), in vacuum crystallizors, either batchwise or continuously, for example in forced-circulation crystallizers (Swenson forced-circulation crystaller) or fluidized-bed crystallizers (Oslo type) (A. D. Randolph, M. A. Larson: Theory of Particulate Processes, 2nd ed., Academic Press, New York 1988; J. Robinson, J. E. Roberts, Can. J. Chem. Eng. 35 (1957) 105-112; J. N\grave{y}vlt: Design of Crystallizers, CRC Press, Boca Raton, 1992). Fractional crystallization is also possible (L. Gordon, M. L. Salutsky, H. H. Willard: Precipitation from Homogeneous Solution, Wiley-Interscience, New York 1959). Likewise, enantiomers and racemates can be separated (J. Jacques, A. Collet, S. H. Willen: Enantiomers, Racemates and Resolutions, Wiley, New York 1981; R. A. Sheldon: Chirotechnology, Marcel Dekker, New York 1993; A. N. Collins, G. N. Sheldrake, J. Crosby (Ed.): Chirality in Industry, Wiley, New York 1985).

Customary filtration methods are, for example, cake filtration and depth filtration (for example described in A. Rushton, A. S. Ward, R. G. Holdich: Solid-Liquid Filtration and Separation Technology, VCH Verlagsgesellschaft, Weinheim 1996, pp. 177ff., K. J. Ives, in A. Rushton (Ed.): Mathematical Models and Design Methods in Solid-Liquid Separation, NATO ASI series E Nr. 88, Martinus Nijhoff, Dordrecht 1985, pp. 90ff.) and cross-flow filtrations, in particular microfiltration for the removal of solids >0.1 μm (for example described in J. Altmann, S. Ripperger, J. Membrane Sci. 124 (1997) 119-128).

In the case of micro- and ultrafiltration, it is possible to employ, for example, microporous (A. S. Michaels: “Ultrafiltration,” in E. S. Perry (ed.): Progress in Separation and Purification, vol. 1, Interscience Publ., New York 1968), homogeneous (J. Crank, G. S. Park (eds.): Diffusion in Polymers, Academic Press, New York 1968; S. A. Stern: “The Separation of Gases by Selective Permeation,” in P. Meares (ed.): Membrane Separation Processes, Elsevier, Amsterdam 1976), asymmetric (R. E. Kesting: Synthetic Polymeric Membranes, A Structural Perspective, Wiley-Interscience, New York 1985) and electrically charged (F. Helfferich: Ion-Exchange, McGraw-Hill, London 1962) membranes which are prepared by a variety of processes (R. Zsigmondy, U.S. Pat. No. 1,421,341, 1922; D. B. Pall, U.S. Pat. No. 4,340,479, 1982; S. Loeb, S. Sourirajan, U.S. Pat. No. 3,133,132, 1964). Typical materials are cellulose esters, nylon, polyvinyl chloride, acrylonitrile, polypropylene, polycarbonate and ceramics. The use of these membranes is accomplished in the form of a plate module (R. F. Madsen, Hyperfiltration and Ultrafiltration in Plate-and-Frame Systems, Elsevier, Amsterdam 1977), spiral module (U.S. Pat. No. 3,417,870, 1968 (D. T. Bray)), tube-bundle or hollow-fiber module (H. Strathmann: “Synthetic Membranes and their Preparation,” in M. C. Porter (ed.): Handbook of Industrial Membrane Technology, Noyes Publication, Park Ridge, N.J. 1990, pp. 1-60). In addition, it is possible to use liquid membranes (N. N. Li: “Permeation Through Liquid Surfactant Membranes,” AlChE J. 17 (1971) 459; S. G. Kimura, S. L. Matson, W. J. Ward III: “Industrial Applications of Facilitated Transport,” in N. N. Li (ed.): Recent Developments in Separation Science, Vol. V, CRC Press, Boca Raton, Fla., 1979, pp. 11-25). The desired substances can either be concentrated on the feed side and discharged via the retentate stream or else depleted on the feed side and discharged via the filtrate/permeate stream.

Customary centrifugation methods are described, for example, in G. Hultsch, H. Wilkesmann, “Filtering Centrifuges,” in D. B. Purchas, Solid-Liquid Separation, Upland Press, Croydon 1977, pp. 493-559; and H. Trawinski, Die äquivalente Klärfläche von Zentrifugen [the equivalent clarifying area of centrifuges], Chem. Ztg. 83 (1959) 606-612. A variety of designs such as tube centrifuges, basket centrifuges and, specifically, pusher centrifuges, slip-filter centrifuges and disk separators may be employed.

In the method according to this first embodiment, the separation of the solid phase from the liquid phase may, if appropriate, be followed by a drying step, which is carried out in the customary manner. Conventional dry methods are described, for example, in O. Krischer, W. Kast: Die wissenschaftlichen Grundlagen der Trocknungstechnik [The scientific basis of drying technology], 3rd ed., Springer, Berlin-Heidelberg-New York 1978; R. B. Keey: Drying: Principles and Practice, Pergamon Press, Oxford 1972; K. Kröll: Trockner und Trocknungsverfahren [Dryers and drying methods], 2nd ed., Springer, Berlin-Heidelberg-New York 1978; Williams-Gardener, A.: Industrial Drying, Houston, Gulf, 1977; K. Kröll, W. Kast: Trocknen und Trockner in der Produktion [Drying and dryers in production], Springer, Berlin-Heidelberg-New York 1989. The examples of drying methods include methods of convective drying, for example in a drying oven, tunnel dryers, belt dryers, disk dryers, jet dryers, fluidized-bed dryers, aerated and rotating drum dryers, spray dryers, pneumatic-convector dryers, cyclone dryers, mixer dryers, paste-grinder dryers, grinder dryers, ring dryers, tower dryers, rotary dryers, carousel dryers. Other methods exploit drying by contact, for example paddle drying vacuum or freeze drying, cone dryers, suction dryers, disk dryers, thin-film contact dryers, drum dryers, viscous-phase dryers, plate dryers, rotary coil dryers, twin-cone dryers; or heat radiation (infrared, for example infra-red rotary dryers) or dielectric energy (microwaves) for the purpose of drying. The drying apparatuses used for thermal drying methods are heated in most cases by steam, oil, gas or electricity and can partly be operated in vacuo, depending on their design.

The liquid phase which has been separated off may be recirculated in the form of process water. The amount of the liquid phase which is not recirculated into the process can be concentrated in a multi-step evaporation process to give a syrup. If the desired metabolite has not been converted from the liquid phase into the solid phase prior to decanting, then the resulting syrup will also comprise the metabolite. As a rule, the syrup has a dry matter content in the range of from 10 to 90% by weight, preferably 20 to 80% by weight and especially preferably 25 to 65% by weight. This syrup is mixed with the solids which are separated off upon decanting and subsequently dried. Drying can be effected for example by means of tumble dryers, spray dryers or paddle dryers, with a tumble dryer preferably being employed. Drying is preferably carried out in such a manner that the solid obtained has a residual moisture content of no more than 30% by weight, preferably no more than 20% by weight, especially preferably no more than 10% by weight and very especially preferably no more than 5% by weight, based on the total dry weight of the solid obtained.

In a second preferred embodiment of the separation of the volatile constituents from the product of value and the nonstarchy solid constituents from the fermentation liquor, the volatile constituents, if appropriate after a previously described preseparation step for solid constituents, are removed by evaporation. Evaporation can be accomplished in a manner known per se. Examples of suitable methods of evaporating volatile constituents are spray drying, fluidized-bed drying or fluidized-bed agglomeration, freeze drying, pneumatic-convector dryers and contact dryers, and extrusion drying. A combination of the abovementioned methods with shape-imparting methods such as extrusion, pelleting or prilling may also be carried out. In the case of these last-mentioned methods, it is preferred to employ partially or largely pre-dried metabolite-containing substance mixtures.

In a particularly preferred embodiment, the removal of the volatile constituents of the fermentation liquor comprises a spray-drying method or a fluidized-bed drying method, including fluidized-bed granulation. To this end, the fermentation liquor, if appropriate after a preliminary separation for removing coarse solids particles, which comprise only small amounts of nonvolatile microbial metabolite, if any, is fed to one or more spray-drying or fluidized-bed drying apparatuses. The transport, or feeding, of the solids-loaded fermentation liquor is expediently carried out by means of customary transport devices for solids-comprising liquids, for example pumps such as eccentric single-rotor screw pumps (for example from Delasco PCM) or high-pressure pumps (for example from LEWA Herbert Ott GmbH).

Spray-drying apparatuses which can be employed are all traditional spray-drying apparatuses known in the art, such as, for example, those described in the above literature, in particular nozzle towers, specifically those equipped with pressurized nozzles, and disk towers; spray dryers with integrated fluidized bed and fluidized-bed spray granulators are preferably employed in the embodiment described hereinbelow, which employs fluidized-bed drying.

Systems which are suitable for drying by means of spray drying are, in particular those in which the solids-loaded fermentation liquor is dried cocurrently or countercurrently, preferably countercurrently. Here, the fermentation liquor is advantageously passed at the head of a vertically arranged spray tower through a nozzle or via a rotating disk into said spray tower and simultaneously atomized, while the stream of gas employed for the drying, for example air or nitrogen, is passed into the spray tower in the upper or lower zone. The volatile constituents of the fermentation liquor are discharged via the lower outlet or via the head of the spray tower, while the nonvolatile or solid constituents, including the desired microbial metabolite, can be discharged, or removed, from the spray tower as essentially dry powder at the bottom and processed further from this step.

However, the desired residual moisture of the products need not be obtained as early as in this one drying step, but can be adjusted in a subsequent, further drying step. To this end, for example a fluidized-bed drying step may follow after the spray-drying step. The waste air of the spray tower and/or the fluidized bed is advantageously freed from entrained particles or dust by means of cyclone and/or filters and collected for further processing; the volatile constituents can then be collected for example in a condensation unit, if appropriate, and reused, for example as recirculated process water.

When designing and operating the apparatus used, a skilled worker will take into consideration the amount of solids in the fermentation liquor, which may be considerable. Thus, in particular the internal diameters and/or discharge ports of spray nozzles employed must be chosen in such a way that the tendency to clog or block is eliminated or kept as low as possible. A suitable size of the discharge ports or of the internal diameter will, as a rule, be around at least 0.4 mm, preferably at least 1 mm and usually, depending on the properties of the fermentation liquor and the substances present therein, of the pressure and of the desired throughput, in the range of from 0.6 to 5 mm.

The stream of gas employed for the drying step usually has a temperature of above the boiling point of the aqueous fermentation liquor at the desired pressure, for example in the range of from 110 to 300° C., in particular from 120 to 250° C. and specifically from 130 to 220° C. It is also possible to warm the aqueous fermentation medium to a temperature of below its boiling point, for example in the range of from 25 to 85° C. and in particular from 30 to 70° C., in order to support the drying process. It is likewise possible to overheat the aqueous fermentation medium above preferably 100° C., the liquid medium being heated to such a point that it does not boil yet before the nozzle under the desired pressure and that spontaneous evaporation takes place after the nozzle has released the pressure.

The fermentation liquor can additionally be mixed with a stream of gas, for example air or nitrogen, which may have been preheated, for example at a temperature in the range of from 30 to 90° C. If dual-substance nozzles are used instead of single-substance nozzles, this mixing step can be accomplished directly before entering the actual drying space of the spray tower.

In any case, when selecting the temperature, the thermal stability, or boiling point, of the desired microbial metabolite is to be taken into consideration. As a rule, it is expedient to adjust the temperature of the stream of gas used for drying to a temperature which is at least 20° C., preferably at least 50° C., lower than the boiling point, or point of decomposition, of the nonvolatile microbial metabolite in question. Here, it must be taken into consideration that the temperature of the drying material can, in some cases, be markedly below the temperature of the added stream of gas, as long as not all of the volatile constituents have been evaporated. In this respect, the temperature of the material to be dried is also influenced via the setting of the residence time. The drying procedure can therefore be carried out at least for some time at inlet-air temperatures which are in the range of the boiling point of the metabolites to be dried or above. The suitable temperature conditions can be determined by the skilled worker by routine experimentation.

In an especially preferred embodiment, the drying process is carried out in a vertically designed spray tower which is operated cocurrently or countercurrently, preferably countercurrently. Feeding the solids-loaded fermentation liquor which has been cooled to room temperature or which still has the fermentation temperature or below, for example from 18° C. to 37° C., is accomplished at the head of the spray tower via one or more, for example 1, 2, 3 or 4, in particular 1 or 2, spray nozzles. The stream of hot gas, preferably air, which is provided for the drying process is passed into the top or bottom zone of the spray tower. The powder obtained is removed at the bottom end or at the head of the spray tower. If desired, this may be followed by fluidized-bed drying.

The mean particle size of the powders obtained is determined largely by the degree of atomization obtained when passing the solids-loaded fermentation liquor into the spray tower. The degree of atomization depends for its part on the pressure used at the spray nozzles or the speed of the rotating disk. The pressure applied at the spray nozzles is usually in the range of from 5 to 200 bar, for example approximately 10 to 100 bar and in particular approximately 20 to 60 bar, above standard pressure. The speed of the rotating disk is usually in the range of from 5000 to 30 000 rpm. The throughput rate of the stream of gas passed in for drying purposes depends greatly on the flow rate of the liquid medium. If the flow rate of the liquid medium is low (for example in the range of from 10 to 1000 l/h), it is usually in the range of from 100 to 10 000 m³/h at higher flow rates (for example in the range of from 1000 to 50 000 l/h) usually in the range of from 10 000 to 10 000 000 m³/h.

If appropriate, customary adjuvants which are known in the art can be used concomitantly with the spray drying process. These adjuvants reduce or prevent agglomeration of the primary powder particles formed in the spray tower so that the properties of the powders discharged from the spray tower can be influenced in the targeted fashion, for example regarding the particle sizes, in the sense of an improved degree of dryness, an improved flowability and/or better redispersibility in solvents such as water. Examples of conventional spray adjuvants are the abovementioned formulation adjuvants. They are employed in the conventionally used quantities, for example in the range of from 0.1 to 50% by weight, in particular 0.1 to 30% by weight and specifically 0.1 to 10% by weight, based on the total dry weight of the nonvolatile solid constituents of the fermentation liquor.

The design expediently to be selected in each case for the apparatus in question, in particular the dimensions of the spray nozzles employed and the suitable operating parameters, can be determined by the skilled worker simply by routine experimentation.

In a further configuration of the second preferred embodiment, the volatile constituents of the fermentation liquor are removed using fluidized-bed drying methods. What has been said above for the use of spray-drying methods also applies here analogously, for example regarding the transport of the solids-containing fermentation liquor, regarding the design of the apparatuses and regarding the choice of operating parameters, in particular the operating temperature. Suitable fluidized-bed drying devices which can be employed are all conventional fluidized-bed dryers known in the art, in particular spray dryers with integrated fluidized bed and fluidized-bed spray granulators, for example from Allgaier, DMR, Glatt, Heinen, Huttlin, Niro and Waldner.

Fluidized-bed dryers can be operated continuously or batchwise. In the case of continuous operation, the residence time in the dryer is from several minutes up to several hours. The apparatus is therefore also suitable for long-retention-time drying, for example over a period of from approximately 1 h to 15 h. If a narrow residence time distribution is desired, the fluidized bed can be divided into cascades, using separation sheets, or the product flow can be approximated to an ideal piston flow by baffles having a meandering design. Larger dryers in particular are divided into a plurality of drying zones, for example 2 to 10 and in particular 2 to 5 drying zones, which are operated at different gas velocities and temperatures. The last zone can then be employed as cooling zone; in this case, an inlet-air temperature in the range of from 10 to 40° C. will usually be set.

In the feed region of the moist material, care will as a rule be taken to avoid agglomerations. This can be accomplished in different ways, for example by a locally higher gas velocity or by employing a stirring mechanism. In the case of smaller systems, or to improve the ease with which the system can be cleaned, the filters for cleaning the waste gas can be integrated in the fluidized-bed dryer.

In the batchwise operated fluidized-bed dryers, the residence time is equally between several minutes and hours. Again, these apparatuses are suitable for long-retention-time drying.

Fluidized-bed dryers can be operated in a vibrating mode, the vibration supporting the product transport at low gas velocities (i.e. below the minimal fluidization velocity) and low bed height and being able to prevent agglomerations. In addition to vibration, a pulsed gas supply can also be employed for reducing the drying-gas consumption. The moist material is mixed with turbulence in the upwardly directed, hot gas stream and thereby dries at high heat and mass transfer coefficients. The gas velocity required depends essentially on the particle size and density. For example, superficial velocities of in the range of from 1 to 10 m/s may be required for particles with diameters of several hundred micrometers. A perforated bottom (perforated plate, conidur plate, bottoms made of woven or sintered metal) prevents the solid from falling into the hot-gas space. Heat is supplied either only via the drying gas, or heat exchangers (tube bundles or plates) are additionally introduced into the fluidized bed (K. Masters: Spray Drying Handbook, Longman Scientific & Technical 1991; Arun S. Mujumdar, Handbook of Industrial Drying, Marcel Dekker, Inc. 1995).

As for the rest, what has been said for spray drying applies analogously to fluidized-bed drying, for example regarding the addition of drying adjuvants and the possibility of influencing the product characteristics in this way.

In the case of oily metabolites, drying by using a fluidized-bed apparatus or a mixer can be effected for example in such a way that an adsorbent is introduced into the fluidized-bed apparatus or mixer and mixed through or fluidized. While doing so, the fermentation liquor with the oily metabolites is sprayed onto the adsorbent. The volatile constituents of the fermentation liquor can then be evaporated by supplying energy to the mixer or are evaporated by the heated stream of air in the fluidized bed.

In a further preferred embodiment, the volatile constituents of the fermentation liquor are removed using freeze-drying methods. Here, the solids-containing fermentation liquor is frozen completely, and the frozen volatile constituents are evaporated from the solid state, i.e. sublimated (Georg-Wilhelm Oetjen, Gefriertrocknen [freeze-drying], VCH 1997). Freeze-drying devices which can be employed are all conventional freeze dryers which are known in the art, for example from Klein Vakuumtechnik and Christ.

In a further preferred embodiment, the volatile constituents of the fermentation liquor are removed using pneumatic-convector dryers. Here, the solids-containing fermentation liquor is applied to the lower section of a vertical drying tube. The drying gas drives the resulting particles upwards at superficial velocities of 10 to 20 m/s. The solids-containing fermentation liquor is charged using screws, spinner disks or pneumatically. The particles are deposited at the head of the drying tube by means of a cyclone and, if the desired degree of drying has not been achieved yet, they can be recirculated into the drying tube or passed into a fluidized bed which is arranged downstream (K. Masters: Spray Drying Handbook, Longman Scientific & Technical 1991; Arun S. Mujumdar, Handbook of Industrial Drying, Marcel Dekker, Inc. 1995). Devices which can be employed are all traditional pneumatic-convector dryers which are known in the art, for example those from Nara and Orth.

In a further preferred embodiment, the volatile constituents of the fermentation liquor are removed using contact dryers. This type of dryer is particularly suitable for drying pasty media. However, the use of contact dryers is also advantageous for those media in which the solids are already present in particulate form. The solids-containing fermentation liquor is applied to the ebullators of the dryer via which the energy is supplied. The volatile constituents of the fermentation liquor evaporate (K. Masters: Spray Drying Handbook, Longman Scientific & Technical 1991; Arun S. Mujumdar, Handbook of Industrial Drying, Marcel Dekker, Inc. 1995). A multiplicity of different designs of contact dryers exists and can be employed, see, in this context, the abovementioned examples. They are known to the skilled worker in particular as: thin-film contact dryer, for example from BUSS-SMS, drum dryers, for example from Gouda, paddle dryers, for example from BTC-Technology and Drais, contact belt dryers, for example from Kunz and Merk, and rotary tube bundle dryers, for example from Vetter.

In a further embodiment of the method according to the invention, where formulation adjuvants are employed before the drying step, it is possible to admix for example stabilizers or binders such as polyvinyl alcohol and gelatin into a suspension of the microbial metabolite, for example in a stirred vessel or before a static mixture. Such a suspension can also be applied to a carrier material, for example by spraying on or mixing in, in a mixer or in a fluidized bed.

A further specific embodiment, where formulation adjuvants are added during the drying step, relates to the powdering of moist drops which comprise the metabolite (see, in this context, EP 0648 076 and EP 835613), where the metabolite-containing suspension is sprayed, and the drops are powdered with a powdering agent, for example silica, starch or one of the abovementioned powdering agents or flow adjuvants, in order to stabilize them, and then likewise dried, for example in a fluidized bed.

In a further specific embodiment, where formulation adjuvants are added after the drying step, relates, for example, to the application of coatings/coating layers to dried particles. In particular flow adjuvants for improving the flow characteristics, for example silica, starches or the other abovementioned flow adjuvants, can be added to the product, both after drying and after the coating step.

To obtain oily metabolites or those with a melting point below the boiling point of water, the product in question is advantageously adsorbed onto an adsorbent (examples see hereinabove). In general, the process is carried out such that the relevant absorbent is added at or after the end of the fermentation of the fermentation liquor. If appropriate, the adsorbent can be added after the fermentation liquor has previously been concentrated. Both hydrophobic and hydrophilic adsorbents can be employed. In the first case, the adsorbents are separated from the volatile constituents of the fermentation liquor together with the adsorbed metabolite in the same manner as the solid constituents together with the latter. In the latter case, care must be taken that the adsorbents, which are in dissolved or suspended form, are not discharged together with the adsorbed products by the processing procedure. When employing filtration, this can be achieved for example by selecting a suitably small pore size of the filters. Preferred hydrophobic or hydrophilic adsorbents are the adsorbents which have been mentioned hereinabove in connection with the preparation of nonvolatile microbial metabolites in pseudosolid form, in particular kieselguhr, silica, sugars and the abovementioned inorganic and organic alkali and alkaline earth metal salts.

A further possibility of product formulation is shaping by mechanical means, for example by means of extrusion, pelleting or what is known as prilling. Here, the metabolite, or the substance mixture comprising it, which has preferably been dried, pre-dried and/or treated with formulation adjuvants, is, as a rule, pushed through a die or a sieve. The product is usually conveyed to the die via one or more screws, an edge runner or other mechanical components, for example rotating or longitudinally moving components. The extrudates obtained after the substance has passed through the die or the sieve can be removed mechanically, for example using a blade, or, if appropriate, disintegrate into smaller particles more or less on their own. Shaping product formulation methods without dies are, for example, compacting and granulating in mixers, for example what is known as high-shear granulation.

The shaping methods mentioned are advantageously employed if, as the result of evaporating a metabolite-containing suspension and/or by adding formulation adjuvants, for example carriers such as starch and adhesives such as lignin or polyvinyl alcohol, to such a suspension, a material is obtainable which is highly viscous, pasty or capable of being granulated and thus being capable of being employed directly in one of these methods. If not, the required highly viscous or pasty consistency can also be obtained by drying or pre-drying the metabolite-containing suspension, for example fermentation liquor, by means of the above-described drying methods, preferably by means of spray drying, before the extrusion, pelleting, compacting, granulation (for example high-shear granulation) or prilling process is carried out. If appropriate, the product obtained in this way is mixed with conventional formulation adjuvants which are known to the skilled worker for this purpose and extruded, pelleted, compacted, granulated or prilled. These methods can also be operated in such a way that at least one constituent of the metabolite-containing substance mixture is melted before the shaping step, and resolidifies after the shaping. As a rule, such an embodiment requires an addition of customary adjuvants which are known to the skilled worker for this purpose. The products obtained here typically have particle sizes in the range of from 500 μm to 0.05 m. Comminution methods such as grinding, if appropriate in combination with screening methods, can, if desired, be used to obtain smaller particle sizes herefrom.

The particles obtained by the shaping formulation methods described can be dried down to the desired residual moisture content by the abovedescribed drying methods.

All of the metabolites obtained in solid form in one of the above-described manners, or substance mixtures comprising them, for example particles, granules and extrudates, can be coated with a coating, i.e. with at least one further substance layer. Coating is effected for example in mixers or in fluidized beds, in which the particles to be coated are fluidized and then sprayed with the coating material. The coating material can be in dry form, for example as a powder, or in the form of a solution, dispersion, emulsion or suspension in a solvent, for example water, organic solvents and mixtures of these, in particular in water. If present, the solvent is removed by evaporation during or after being sprayed onto the particles. Moreover, coating materials such as fats may also be applied in the form of melts.

Coating materials which can be sprayed on in the form of an aqueous dispersion or suspension are described for example in WO 03/059087. These include, in particular, polyolefins such as polyethylene, polypropylene, polyethylene waxes, waxes, salts such as alkali or alkaline earth metal sulfates, alkali or alkaline earth metal chlorides and alkali or alkaline earth metal carbonates, for example sodium sulfate, magnesium sulfate, calcium sulfate, sodium chloride, magnesium chloride, calcium chloride, sodium carbonate, magnesium carbonate and calcium carbonate; acronals, for example butyl acrylate/methyl acrylate copolymer, the Styrofan brands from BASF, for example based on styrene and butadiene, and hydrophobic substances as described in WO 03/059086. When applying such materials, the solids content of the coating material is typically in the range of from 0.1 to 30% by weight, in particular in the range of from 0.2 to 15% by weight and specifically in the range of from 0.4 to 5% by weight, in each case based on the total weight of the formulated end product.

Coating materials which can be sprayed in the form of solutions are, for example, polyethylene glycols, cellulose derivatives such as methylcellulose, hydroxypropyl-methylcellulose and ethylcellulose, polyvinyl alcohol, proteins such as gelatin, salts such as alkali or alkaline earth metal sulfates, alkali or alkaline earth metal chlorides and alkali or alkaline earth metal carbonates, for example sodium sulfate, magnesium sulfate, calcium sulfate, sodium chloride, magnesium chloride, calcium chloride, sodium carbonate, magnesium carbonate and calcium carbonate; carbohydrates such as sugars, for example glucose, lactose, fructose, sucrose and trehalose; starches and modified starches. When applying such materials, the solids content of the coating material is typically in the range of from 0.1 to 30% by weight, in particular in the range of from 0.2 to 15% by weight and specifically in the range of from 0.4 to 10% by weight, in each case based on the total weight of the formulated end product.

Coating materials which can be sprayed on in the form of a melt are described, for example, in DE 199 29 257 and WO 92/12645. These include, in particular, polyethylene glycols, synthetic fats and waxes, for example Polygen WE® from BASF, natural fats such as animal fats, for example beeswax, and vegetable fats, for example candelilla wax, fatty acids, for example animal waxes, tallow fatty acids, palmitic acid, stearic acid, triglycerides, Edenor products, Vegeole products, montan ester waxes, for example Luwax E® from BASF. When applying such materials, the solids content of the coating material is typically in the range of from 1 to 30% by weight, in particular in the range of from 2 to 25% by weight and specifically in the range of from 3 to 20% by weight, in each case based on the total weight of the formulated end product.

Coating materials which can be used as powders in the dry-coating process are, for example, polyethylene glycols, cellulose and cellulose derivatives such as methylcellulose, hydroxypropylmethylcellulose and ethylcellulose, polyvinyl alcohol, proteins such as gelatin, salts such as alkali and alkaline earth metal sulfates, alkali and alkaline earth metal chlorides and alkali or alkaline earth metal carbonates, for example sodium sulfate, magnesium sulfate, calcium sulfate, sodium chloride, magnesium chloride, calcium chloride, sodium carbonate, magnesium carbonate and calcium carbonate; carbohydrates such as sugars, for example glucose, lactose, fructose, sucrose and trehalose, starches and modified starches, fats, fatty acids, tallow, flour, for example of maize, wheat, rye, barley or rice, clay, ash and kaolin. The adhesion between the powder to be applied as the coating and the products to be coated can be accomplished with the substances which can be sprayed on in the form of solutions or melts. The spraying of these solutions or melts can be effected alternately with the introduction of the powder or else in parallel. Preferably, the product to be coated is fluidized in a fluidized bed or a mixer. The powder is then conveyed, preferably continuously, into the fluidized bed or the mixer in order to be coated. In an especially preferred embodiment, the process space is charged with the solution or melt while adding the powder. The solution can be supplied for example via a connection piece or, preferably, sprayed into the process space via a nozzle (for example single-substance or dual-substance nozzle). It is especially preferred that the feeding station of the powder and the position of the nozzle in the process space are spatially separate from one another so that the solution or melt comes predominantly into contact with the product to be coated and not the powder to be applied.

It is also possible to apply mixtures of different coating materials, in particular, it is possible to apply a plurality of identical or different coating layers in succession.

In an alternative embodiment, the desired nonvolatile microbial metabolite can be obtained from the remaining fermentation liquor together with the solid constituents of the fermentation liquor, analogously to the by-product obtained in the bioethanol production (where it is called “Distiller's Dried Grains with Solubles (DDGS)” and marketed as such). In this case, essentially all, or only some, of the liquid constituents of the fermentation liquor can be removed from the solids. The proteinaceous by-product obtained in this manner can be used as feed or feed additive for feeding animals, preferably agricultural livestock, especially preferably cattle, pigs and poultry, very especially preferably cattle, either before or after further working or processing steps.

To this end, usually all of the liquor, i.e. including the nonvolatile microbial metabolite and the other insoluble or solid constituents, is concentrated (evaporated) to a certain degree in an evaporation procedure which is a single-step or, as a rule, a multi-step evaporation procedure, and the solids comprised are subsequently removed from the remaining liquid (liquid phase), for example using a decanter. In the method according to the invention, the desired metabolite can first be converted from the liquid phase into the solid form, for example by crystallization or precipitation, so that it is obtained together with the other solids. The solids which are removed here generally have a dry-matter content in the range of from 10 to 80% by weight, preferably 15 to 60% by weight and especially preferably 20 to 50% by weight and can, if appropriate, be dried further using customary drying methods, for example those described hereinabove. The finished formulation obtained by further working or processing advantageously has a dry matter content of at least approximately 90%, so that the risk of spoilage upon storage is reduced.

The liquid phase which has been separated off can be recirculated as process water. The portion of the liquid phase which is not recirculated into the process can be concentrated in a multi-step evaporation process to give a syrup. If the desired metabolite has not been converted from the liquid into the solid phase before the decanting step, then the resulting syrup will also comprise the metabolite. As a rule, the syrup has a dry matter content in the range of from 10 to 90% by weight, preferably 20 to 80% by weight and especially preferably 25 to 65% by weight. This syrup is mixed with the solids which have been separated upon decanting and subsequently dried. Drying can be effected for example by means of drum dryer, spray dryer or paddle dryer, a drum dryer is preferably employed. Drying is preferably carried out in such a way that the solid obtained has a residual moisture content of not more than 30% by weight, preferably not more than 20% by weight, especially preferably not more than 10% by weight and very especially preferably not more than 5% by weight, based on the total dry weight of the solid obtained.

Not only the liquid phase separated off in this alternative embodiment can be recirculated as process water, but also the volatile constituents which may have been collected in the other, above-described embodiments after having undergone condensation. These recirculated portions of the liquid or volatile phase can advantageously, for example fully or in part, be employed in the production of the sugar-containing liquid of step a) or used for making up buffer or nutrient salt solution for use in the fermentation. When admixing recirculated process water in step a), it must be taken into consideration that an unduly high percentage may have an adverse effect on the fermentation as the result of the unduly high supply of certain mineral substances and ions, for example sodium and lactate ions. Preferably, the percentage of recirculated process water when making up the suspension for the starch liquefaction is therefore limited according to the invention to not more than 75% by weight, preferably not more than 60% by weight and especially preferably not more than 50% by weight. The percentage of process water when making up the suspension in the preferred embodiment of step a2) is advantageously in the range of from 5 to 60% by weight and preferably 10 to 50% by weight.

As the result of the drying and confectioning methods described herein, the mean particle sizes of the solids obtained can be varied within a substantial range, for example from relatively small particles in the range of from approximately 1 to 100 μM via medium particle sizes in the range of from 100 up to several hundred μm up to relatively large particles of approximately at least 500 μm or approximately 1 mm and larger up to several mm, for example up to 10 mm. In the preparation of powders, the mean particle size is, as a rule, in the range of from 50 to 1000 μm. In the preparation of other solid forms of the products, for example extrudates, compactates and in particular granules, prepared, for example, by fluidized-bed spray dryers and spray granulators, larger dimensions will, as a rule, be set, the mean particle size frequently being in the range of from 200 to 5000 μm. The term “mean particle size” here refers to the average of the maximum particle lengths of the individual particles in the case of nonspherical particles, or to the average of the diameters of spherical or nearly spherical particles. It must be taken into consideration that larger secondary particles can be formed during the spray-drying process as the result of agglomeration of the primary particles. Carrying out the method according to the invention gives the particle size distributions conventionally obtained in spray drying.

The invention furthermore relates to a method as described above, wherein
(i) a portion of not more than 50% by weight is removed from the sugar-containing liquid medium obtained in step a2), which comprises the nonstarchy solid constituents of the starch feedstock selected from cereal kernels, and the remainder is used to carry out a fermentation for the production of a first nonvolatile metabolite (A), in solid form; and
(ii) all or some of the nonstarchy solid constituents of the starch feedstock are removed from this portion, which is used to carry out a fermentation for the production of a second nonvolatile metabolite (B) in solid form, which is identical to, or different from, the metabolite (A).

In a preferred embodiment, the nonstarchy solid constituents of (ii) are separated off in such a way that the solids content of the remaining portion of the sugar-containing liquid medium amounts to preferably not more than 50% by weight, preferably not more than 30% by weight, especially preferably not more than 10% by weight and very especially preferably not more than 5% by weight.

This procedure makes possible, in the separate fermentation of (ii), the use of microorganisms for which certain minimum requirements, for example with regard to the oxygen transfer rate, must be met. Suitable microorganisms which are employed in the separate fermentation of (ii) are, for example, Bacillus species, preferably Bacillus subtilis. The compounds produced by such microorganisms in the separate fermentation are selected in particular from vitamins, cofactors and nutraceuticals, purine and pyrimidine bases, nucleosides and nucleotides, lipids, saturated and unsaturated fatty acids, aromatic compounds, proteins, carotenoids, specifically from vitamins, cofactors and nutraceuticals, proteins and carotenoids, and very specifically from riboflavin and calcium pantothenate.

A preferred embodiment of this procedure relates to parallel production of identical metabolites (A) and (B) in two separate fermentations. This is advantageous in particular in a case where different applications of the same metabolite have different purity requirements. Accordingly, the first metabolite (A), for example an amino acid to be used as food additive, for example lysine, is produced using the solids-containing fermentation liquor and the same second metabolite (B), for example the same amino acid to be used as food additive, in the present case for example lysine, is produced using the fermentation liquor which has been solids-depleted in accordance with (ii). Owing to the complete or partial removal of the nonstarchy solid constituents, the complexity of the purification when working up the metabolite whose field of application has a higher purity requirement, for example as food additive, can be reduced.

In a further preferred embodiment of this procedure, the metabolite B produced by the microorganisms in the fermentation is riboflavin. To carry out the fermentation, analogous conditions and procedures as have been described for other carbon feedstocks, for example in WO 01/011052, DE 19840709, WO 98/29539, EP 1186664 and Fujioka, K.: New biotechnology for riboflavin (vitamin B2) and character of this riboflavin. Fragrance Journal (2003), 31 (3), 44-48, can be employed.

To carry out this variant of the process, the following procedure may be used, for example. A preferably large-volume fermentation is implemented for the production of metabolites A, for example of amino acids such as lysine, in accordance with the method according to the invention, for example using the preferred process steps a) to c). In accordance with (i), some of the sugar-containing liquid medium obtained in step a) is removed and freed in accordance with (ii) completely or in part from the solids by customary methods, for example centrifugation or filtration. The sugar-containing liquid medium obtained therefrom, which is essentially fully or partially freed from the solids, is, in accordance with (ii), fed to a fermentation for the production of a metabolite B, for example riboflavin. The solids stream separated in accordance with (ii) is advantageously returned to the stream of the sugar-containing liquid medium of the large-volume fermentation.

The riboflavin-containing fermentation liquor which is thus generated in accordance with (ii) can be worked up by analogous conditions and procedures as have been described for other carbon feedstocks, for example in DE 4037441, EP 464582, EP 438767 and DE 3819745. Following lysis of the cell mass, the riboflavin, which is present in crystalline form, is separated, preferably by decanting. Other ways of separating solids, for example filtration, are also possible. Thereafter, the riboflavin is dried, preferably by means of spray dryers and fluidized-bed dryers. As an alternative, the riboflavin-containing fermentation mixture produced in accordance with (ii) can be processed under analogous conditions and using analogous procedures as described in, for example, EP 1048668 and EP 730034. After pasteurization, the fermentation liquor is centrifuged here, and the remaining solids-containing fraction is treated with a mineral acid. The riboflavin formed is removed from the aqueous-acidic medium by filtration, washed, if appropriate, and subsequently dried.

In a further preferred embodiment of this procedure, the metabolite B produced by the microorganisms in the fermentation is pantothenic acid. To carry out the fermentation, analogous conditions and procedures as have been described for other carbon feedstocks, for example in WO 01/021772, can be employed.

To carry out this process variant, a procedure such as described above for riboflavin may be followed for example. The sugar-containing liquid medium which has been subjected to a preliminary purification in accordance with (ii) and which has preferably been essentially freed from the solids is fed to a fermentation in accordance with (ii) for the production of pantothenic acid. Here, the fact that the viscosity is reduced in comparison with the solids-containing liquid medium is particularly advantageous. The separated solids stream is preferably returned to the stream of the sugar-containing liquid medium of the large-volume fermentation.

The pantothenic-acid-containing fermentation liquor produced in accordance with (ii) can be worked up under analogous conditions and using analogous procedures as have been described for other carbon feedstocks, for example in EP 1 050 219 and WO 01/83799. After all of the fermentation liquor has been pasteurized, the remaining solids are separated, for example by centrifugation or filtration. The clear runoff obtained in the solids separation step is partly evaporated, if appropriate treated with calcium chloride and dried, in particular spray dried.

The solids which have been separated off are obtained together with the respective desired nonvolatile microbial metabolite (A) within the scope of the parallel large-volume fermentation process.

After the drying and/or formulation step, whole or ground cereal kernels, preferably maize, wheat, barley, millet/sorghum, triticale and/or rye, may be added to the product formulation.

The invention furthermore relates to solid formulations of nonvolatile metabolites which can be obtained by the method described herein. In addition to the at least one nonvolatile metabolite (constituent A) of the fermentation, the formulations usually comprise biomass from the fermentation (constituent B) and some or all of the nonstarchy solid constituents of the starch feedstock (constituent C). In addition, the substance mixtures according to the invention further comprise if appropriate the abovementioned formulation adjuvants such as binders, carriers, powdering/flow adjuvants, film or color pigments, biocides, dispersants, antifoam agents, viscosity regulators, acids, bases, antioxidants, enzyme stabilizers, enzyme inhibitors, adsorbates, fats, fatty acids, oils and the like.

The metabolite typically amounts to more than 10% by weight, for example >10 to 80% by weight, in particular 20 to 60% by weight, based on the total amount of the components A, B and C. Based on the total weight of the formulation, the metabolite typically amounts to 0.5 to 80% by weight, in particular 1 to 60% by weight, based on the total weight of the formulation.

The biomass from the fermentation which produces the nonvolatile metabolite typically amounts to 1 to 50% by weight, in particular 10 to 40% by weight, based on the total amount of the components A, B and C, or 0.5 to 50% by weight, in particular 2 to 40% by weight, based on the total weight of the formulation.

As a rule, the nonstarchy solid constituents of the starch feedstock from the fermentation liquor amounts to at least 1% by weight and in particular 5 to 50% by weight, based on the total amount of the components A, B and C, or at least 0.5% by weight, in particular at least 2% by weight, for example in the range of from 2 to 50% by weight, in particular 5 to 40% by weight, based on the total weight of the formulation.

As a rule, the formulation adjuvants will amount to up to 400% by weight, based on the total weight of the components A, B and C, frequently in the range of from 0 to 100% by weight, based on the total amount of the components A, B and C, or in the range of from 0 to 80 and in particular 1 to 30% by weight, based on the total weight of the formulation.

The formulations according to the invention are in solid form, typically in the form of powders, granules, pellets, extrudates, compactates or agglomerates.

The formulations according to the invention typically contain dietary fibers which result firstly from the solid constituents of the starch feedstock and which are furthermore employed as extenders/carriers in the preparation of the formulations according to the invention. As regards the definition of the components which come under the term “dietary fibers” for the purposes of the invention, reference is made to the report of the American Association of Cereal Chemists (AACC) in Cereal Foods World (CFW), 46 (3), “The Definition of Dietary Fiber”, 2001, pp. 112-129, in particular pp. 112, 113 and 118. As a rule, the dietary fibers amount to at least 1% by weight, in particular at least 5% by weight, specifically at least 10% by weight and frequently in the range of from 1 to 60% by weight, in particular 5 to 50% by weight and specifically in the range of from 10 to 40% by weight, in each case based on the total weight of the formulation. As a rule, the dietary fiber content is determined by an AACC standard method (American Association of Cereal Chemists. 2000. Approved Methods of the American Association of Cereal Chemists, 10^{th} ed., Method 32-25, Total dietary fiber determined as neutral sugar residues, uronic acid residues, and Klason lignin (Uppsala method). The Association, St. Paul, Minn.).

The substance mixtures according to the invention have a high protein content which corresponds essentially to the biomass B. Further portions of the protein content can also originate from the starch feedstock employed. The protein content is typically in the range of from 20 to 70% by weight based on the total weight of the formulation.

The inherent protein content (specifically component B) and dietary fiber content (specifically component C) is advantageous for a variety of formulation methods, for example in the case of oily metabolites, in particular in view of drying steps employed in this context.

The formulations according to the invention advantageously comprise one or more essential amino acids, in particular at least one amino acid selected among lysine, methionine, threonine and tryptophan. If present, the essential amino acids, in particular those mentioned, are, as a rule, each present in an amount which is increased over a traditional DDGS by-product which is generated in a fermentative bioethanol production, in particular by a factor of at least 1.5. If the amino acid in question is present in the formulation, the formulation has, as a rule, a lysine content of at least 1% by weight, in particular in the range of from 1 to 10% by weight and specifically in the range of from 1 to 5% by weight, a methionine content of at least 0.8% by weight, in particular in the range of from 0.8 to 10% by weight and specifically in the range of from 0.8 to 5% by weight, a threonine content of at least 1.5% by weight, in particular in the range of from 1.5 to 10% by weight, and specifically in the range of from 1.5 to 5% by weight, and/or a tryptophan content of at least 0.4% by weight, in particular in the range of from 0.4 to 10% by weight and specifically in the range of from 0.4 to 5% by weight, in each case based on the total dry matter of the formulation.

The formulations according to the invention conventionally also comprise a small amount of water, frequently in the range of from 0 to 25% by weight, in particular in the range of from 0.5 to 15% by weight, specifically in the range of from 1 to 10% by weight and very specifically in the range of from 1 to 5% by weight of water, in each case based on the total weight of the formulation.

The formulations according to the invention are suitable for use in animal or human nutrition, for example as such or as additive or supplement, also in the form of premixes. Suitable for this purpose are, in particular, formulations which comprise amino acids, for example lysine, glutamate, methionine, phenyalanine, threonine or tryptophan; vitamins, for example vitamin B₂ (riboflavin), vitamin B₆ or vitamin B₁₂; carotenoids, for example astaxanthin or cantaxanthin; sugars, for example trehalose; or organic acids, for example fumaric acid.

The formulations according to the invention are also suitable for use in the textile, leather, cellulose and paper industries. Formulations employed in particular in the textile sector are those which comprise enzymes such as amylases, pectinases and/or acid, hybrid or neutral cellulases as metabolites; in the leather sector in particular those which comprise enzymes such as lipases, pancreases or proteases; and in the cellulose and paper industries in particular those which comprise enzymes such as amylases, xylanases, cellulases, pectinases, lipases, esterases, proteases, oxidoreductases, for example laccase, catalase and peroxidase.

The examples which follow are intended to illustrate individual aspects of the present invention but are in no way to be understood as being limiting.

### EXAMPLES

### I. Milling the Starch Feedstock

The millbases employed hereinbelow were produced as follows. Whole maize kernels were fully milled using a rotor mill. Using different beaters, milling paths or screen elements, three different degrees of fineness were obtained. A screen analysis of the millbase by means of a laboratory vibration screen (vibration analyzer: Retsch Vibrotronic type VE1; screening time 5 minutes, amplitude: 1.5 mm) gave the results listed in Table 1.

TABLE 1

| Experiment number | | T 70/03 | T 71/03 | T 72/03 |
|---|---|---|---|---|
| <2 | mm/%¹⁾ | 99.4 | 100 | 100 |
| <0.8 | mm/% | 66 | 100 | 99 |
| <0.63 | mm/% | 58.6 | 98.5 | 91 |
| <0.315 | mm/% | 48.8 | 89 | 65 |
| <0.1 | mm/% | | 25 | 9.6 |
| <0.04 | mm/% | | 8 | 3.2 |

| | | | | | | |
|---|---|---|---|---|---|---|
| Millbase in total | 20 | kg | 11.45 | kg | 13.75 | kg |

¹⁾% by weight based on the total amount of millbase

### II. Enzymatic Starch Liquefaction and Starch Saccharification

### II.1. Without Phytase in the Saccharification Step

### II.1a) Enzymatic Starch Liquefaction

320 g of dry-milled maize meal (T71/03) were suspended in 480 g of water and admixed with 310 mg of calcium chloride with continuous stirring. Stirring was continued during the entire experiment. After the pH was brought to 6.5 with H₂SO₄ and the mixture had been heated to 35° C., 2.4 g of Termamyl 120L type L (Novozymes A/S) were added. In the course of 40 minutes, the reaction mixture was heated to a temperature of 86.5° C., the pH being readjusted with NaOH to the above value, if appropriate. Within 30 minutes, a further 400 g of the dry-milled maize meal (T71/03) were added, during which process the temperature was raised to 91° C. The reaction mixture was held at this temperature for approximately 100 minutes. A further 2.4 g of Termamyl 120L were subsequently added and the temperature was held for approximately 100 minutes. The progress of the liquefaction was monitored during the experimentation using the iodine-starch reaction. The temperature was finally raised to 100° C. and the reaction mixture was boiled for a further 20 minutes. At this point in time, starch was no longer detectable. The reactor was cooled to 35° C.

### II.1b) Saccharification

The reaction mixture obtained in II.1a) was heated to 61° C., with constant stirring. Stirring was continued during the entire experiment. After the pH had been brought to 4.3 with H₂SO₄, 10.8 g (9.15 ml) of Dextrozyme GA (Novozymes A/S) were added. The temperature was held for approximately 3 hours, during which time the progress of the reaction was monitored with glucose test strips (S-Glucotest by Boehringer). The results are listed in Table 2 hereinbelow. The reaction mixture was subsequently heated to 80° C. and then cooled. This gave approximately 1180 g of liquid product with a density of approximately 1.2 kg/l and a dry matter content which, as determined by infrared dryer, amounted to approximately 53.7% by weight. After washing with water, a dry matter content (without water-soluble constituents) of approximately 14% by weight was obtained. The glucose content of the reaction mixture, as determined by HPLC, amounted to 380 g/l (see Table 2, sample No. 7).

TABLE 2

| | min (from addition | Glucose concentration |
|---|---|---|
| Sample No. | of glucoamylase) | in supernatant [g/l] |
| 1 | 5 | 135 |
| 2 | 45 | 303 |
| 3 | 115 | 331 |
| 4 | 135 | 334 |
| 5 | 165 | 340 |
| 6 | 195 | 359 |
| 7 | 225 | 380 |

### II.2. With Phytase in the Saccharification Step

### II.2a) Starch Liquefaction

A dry-milled maize meal sample is liquefied as described in II.1a).

### II.2b) Saccharification

The reaction mixture obtained in II.2a) is heated to 61° C. with constant stirring. Stirring is continued during the entire experiment. After the pH has been brought to 4.3 with H₂SO₄, 10.8 g (9.15 ml) of Dextrozyme GA (Novozymes A/S) and 70 μl of phytase (700 units of phytase, Natuphyt Liquid 10 000L from BASF AG) are added. The temperature is held for approximately 3 hours, during which time the progress of the reaction is monitored with glucose test strips (S-Glucotest by Boehringer). The reaction mixture is subsequently heated to 80° C. and then cooled. The product obtained is dried by infrared dryer and washed with water. The glucose content of the reaction mixture is determined by HPLC.

### II.3 Further Protocols for the Enzymatic Liquefaction and Saccharification of Starch

### II.3a) Maize Meal

360 g of deionized water are introduced into a reaction vessel. 1.54 ml of CaCl₂ stock solution (100 g CaCl₂×2H₂O/l) are added to the slurry to a final concentration of approximately 70 ppm Ca²⁺. 240 g of maize meal are slowly run into the water, with constant stirring. After the pH has been brought to 6.5 using 50% by weight strength aqueous NaOH solution, 4.0 ml (=2% by weight enzyme/dry matter) of Termamyl 120 L type L (Novozymes A/S) are added. The slurry is then heated rapidly up to 85° C. During this process, it is necessary to constantly monitor and, if appropriate, adjust the pH.

After the final temperature has been reached, the addition of further meal is commenced, initially 50 g of meal. In addition, 0.13 ml of CaCl₂ stock solution is added to the slurry in order to maintain the Ca²⁺ concentration at 70 ppm. During the addition, the temperature is held at a constant 85° C. At least 10 minutes are allowed to pass in order to ensure a complete reaction before a further portion (50 g of meal and 0.13 ml of CaCl₂ stock solution) are added. After the addition of two portions, 1.67 ml of Termamyl are added; thereafter, two further portions (in each case 50 g of meal and 0.13 ml of CaCl₂ stock solution) are added. A dry-matter content of 55% by weight is reached. After the addition, the temperature is raised to 100° C., and the slurry is boiled for 10 minutes.

A sample is taken and cooled to room temperature. After the sample has been diluted with deionized water (approximately 1:10), one drop of concentrated Lugol's solution (mixture of 5 g of I and 10 g of KI per liter) is added. An intense blue coloration indicates that residual starch is present; a brown coloration is observed when all of the starch has been hydrolyzed. When the test indicates that a portion of residual starch is present, the temperature is again lowered to 85° C. and kept constant. A further 1.67 ml of Termamyl are added until the iodine-starch reaction is negative.

For the subsequent saccharification reaction, the mixture, which tests negative for starch, is brought to 61° C. The pH is brought to 4.3 by addition of 50% strength sulfuric acid. In the course of the reaction, the pH is maintained at this value. The temperature is maintained at 61° C. 5.74 ml (=1.5% by weight enzyme/dry matter) of Dextrozym GA (Novozymes A/S) are added in order to convert the liquefied starch into glucose. The reaction is allowed to proceed for one hour. To inactivate the enzyme, the mixture is heated to 85° C. The hot mixture is filled into sterile containers, which are cooled and then stored at 4° C. A final glucose concentration of 420 g/l was obtained.

II.3b) Rye Meal (Including Pretreatment with Cellulase/Hemicellulase)

360 g of deionized water are introduced into a reaction vessel. 155 g of rye meal are slowly run into the water, with constant stirring. The temperature is maintained at a constant 50° C. After the pH has been brought to 5.5 using 50% by weight strength of aqueous NaOH solution, 3.21 ml (=2.5% by weight enzyme/dry matter) of Viscozyme L (Novozymes A/S) are added. After 30 minutes, the addition of further meal is started, with 55 g of meal being added initially. After a further 30 minutes, a further 50 g of meal are added; 30 minutes later, a further 40 g of meal are added. 30 minutes after the last addition, the liquefaction may be started.

1.7 ml of CaCl₂ stock solution (100 g CaCl₂×2H₂O/1) are added. After the pH has been adjusted to 6.5 using 50% by weight of aqueous NaOH solution, 5.0 ml (=2% by weight enzyme/dry matter) of Termamyl 120 L type L (Novozymes A/S) are added. The slurry is then heated rapidly at 85° C. During this process, the pH is continuously monitored and, if appropriate, adjusted.

After the final temperature has been reached, the addition of further meal is commenced, initially 60 g of meal. In addition, 0.13 ml of CaCl₂ stock solution is added to the slurry in order to maintain the Ca²⁺ concentration at 70 ppm. During the addition, the temperature is held at a constant 85° C. At least 10 minutes are allowed to pass in order to ensure a complete reaction before a further portion (40 g of meal and 0.1 ml of CaCl₂ stock solution) is added. 1.1 ml of Termamyl are added; thereafter, a further portion (40 g of meal and 0.1 ml of CaCl₂ stock solution) is added. A dry-mass content of 55% by weight is reached. After the addition, the temperature is raised to 100° C., and the slurry is boiled for 10 minutes.

A sample is taken and cooled to room temperature. After the sample has been diluted with deionized water (approximately 1:10), one drop of concentrated Lugol's solution (mixture of 5 g of I and 10 g of KI per liter) is added. An intense blue coloration indicates that residual starch is present; a brown coloration is observed when all of the starch has been hydrolyzed. When the test indicates that a portion of residual starch is present, the temperature is again lowered to 85° C. and kept constant. A further 1.1 ml of Termamyl are added until the iodine-starch reaction is negative.

For the subsequent saccharification reaction, the mixture, which tests negative for starch, is brought to 61° C. The pH is brought to 4.3 by addition of 50% strength sulfuric acid. In the course of the reaction, the pH is maintained at this value. The temperature is maintained at 61° C. 5.74 ml (=1.5% by weight enzyme/dry matter) of Dextrozym GA (Novozymes A/S) are added in order to convert the liquefied starch into glucose. The reaction is allowed to proceed for one hour. To inactivate the enzyme, the mixture is heated at 85° C. The hot mixture is filled into sterile containers, which are cooled and then stored at 4° C. A final glucose concentration of 370 g/l was obtained.

II.3c) Wheat Meal (Including Pretreatment with Xylanase)

360 g of deionized water are introduced into a reaction vessel. The water is heated to 55° C., and the pH is adjusted to 6.0 using 50% by weight strength aqueous NaOH solution. After the temperature and the pH have been adjusted, 3.21 ml (=2.5% by weight enzyme/dry matter) of Shearzyme 500L (Novozymes A/S) are added. 155 g of wheat meal are slowly run into the solution, with constant stirring. The temperature and the pH are kept constant. After 30 minutes, the addition of further meal is started, with 55 g of meal being added initially. After a further 30 minutes, a further 50 g of meal are added; 30 minutes later, a further 40 g of meal are added. 30 minutes after the last addition, the liquefaction may be started.

The liquefaction and saccharification are carried out as described in 11.3b. A final glucose concentration of 400 g/l was obtained.

### III. Strain ATCC13032 lysC^{fbr}

In some of the examples which follow, a modified Corynebacterium glutamicum strain, which has been described in WO 05/059144 under the name ATCC13032 lysC^{fbr} was employed.

### Example 1

### a) Enzymatic Starch Liquefaction and Saccharification

500 g of dry-milled maize meal were suspended in 750 ml of water and again milled finely in a stirred mixer. The suspension was divided into 4 samples Nos. 1 to 4, and each of which was treated with approximately 3 g of thermally stable α-amylase (samples Nos. 1 and 2: Termamyl L; samples Nos. 3 and 4: Spezyme). Samples Nos. 2 and 4 were then treated with approximately 7 g/l glucoamylase (sample No. 2: Dextrozyme GA; sample No. 4: Optidex). This gave pale yellow viscous samples whose solids content was in each case separated off by centrifugation, a layer of hydrophobic solids floating above the clear liquid phase.

Ignoring or taking into consideration the pellet which had been centrifuged off, the clear supernatant of each of the samples obtained in this way was analyzed in concentrated form and after 10-fold dilution, using HPLC. When the pellet was taken into consideration, a pellet dry-matter content of 50% by weight was assumed. The results based on the original sample are listed in Table 3 hereinbelow.

TABLE 3

Sample No.

| | | | |
|---|---|---|---|
| 1 | 2 | 3 | 4 |

Supernatant, 10-fold dilution, without pellet

| | | | | |
|---|---|---|---|---|
| Glucose [g/kg] | 73.0 | 287.3 | 63.7 | 285.1 |
| Fructose [g/kg] | 3.4 | 2.3 | 5.3 | 2.7 |
| Oligosaccharides [g/kg] | 202.1 | 38.2 | 150.8 | 31.5 |
| Total sugars [g/kg] | 278 | 328 | 220 | 319 |

Supernatant, 10-fold dilution, with pellet

| | | | | |
|---|---|---|---|---|
| Glucose [g/kg] | | 178 | | 168 |
| Total sugars [g/kg] | 172 | 203 | 130 | 188 |

Supernatant, without dilution, with pellet

| | | |
|---|---|---|
| Glucose [g/kg] | 198 | 189 |

### b) Fermentation

Two maize meal hydrolyzates obtained in accordance with Example II. 1 were employed in shake-flask experiments using Corynebacterium glutamicum (flasks 4-9). In addition, a wheat meal hydrolyzate prepared analogously to Example II.1 was used in parallel (flasks 1-3).

### b.1) Preparation of the Inoculum

The cells are streaked onto sterile CM agar (composition: see Table 4; 20 minutes at 121° C.) and then incubated for 48 hours at 30° C. The cells are subsequently scraped from the plates and resuspended in saline. 25 ml of the medium (see Table 5) in 250 ml Erlenmeyer flasks are inoculated in each case with such an amount of the cell suspension thus prepared that the optical density reaches an OD₆₀₀ value of 1 at 600 nm.

TABLE 4
Composition of the CM agar plates

| Concentration | Constituent |
|---|---|
| 10.0 g/l | D-glucose |
| 2.5 g/l | NaCl |
| 2.0 g/l | Urea |
| 10.0 g/l | Bacto peptone (Difco) |
| 5.0 g/l | Yeast extract (Difco) |
| 5.0 g/l | Beef extract (Difco) |
| 22.0 g/l | Agar |

### b.2) Preparation of the Fermentation Liquor

The compositions of the flask media 1 to 9 are listed in Table 5.

TABLE 5
Flask media

Flask No.

| | | 1-3 | 4-6 | 7-9 |
|---|---|---|---|---|
| Wheat | 399.66 g/kg** | 250 g/l*** | | |
| Maize I | 283.21 g/kg** | | 353 g/l*** | |
| Maize II | 279.15 g/kg** | | | 358 g/l*** |

| | | |
|---|---|---|
| (NH₄)₂SO₄ | 50 | g/l |
| MgSO₄•7H₂O | 0.4 | g/l |
| KH₂PO₄ | 0.6 | g/l |
| FeSO₄•7H₂O | 2 | mg/l |
| MnSO₄•H₂O | 2 | mg/l |
| Thiamine HCl | 0.3 | mg/l |
| Biotin | 1 | mg/l |
| CaCO₃ | 50 | g/l |

| | |
|---|---|
| pH* | 7.8 |

*to be adjusted with dilute aqueous NaOH solution
**glucose concentration in the hydrolyzate
***amount of weighed-in hydrolyzate per liter of medium

After the inoculation, the flasks were incubated for 48 hours at 30° C. and with shaking (200 rpm) in a humidified shaker. After the fermentation was terminated, the sugar and lysine content was determined by HPLC. The HPLC was carried out using a 1100 Series LC System from Agilent. Pre-column derivatization with ortho-phthalaldehyde permits the quantitative determination of the amino acid formed, the product mixture is separated using a Hypersil AA column from Agilent. The results are compiled in Table 6.

TABLE 6

| Flask | Fructose | Glucose | Sucrose | Total sugars |
|---|---|---|---|---|
| No. | g/l | g/l | g/l | g/l |
| 1 | 0.00 | 0.00 | 4.71 | 4.71 |
| 2 | 0.00 | 7.75 | 4.82 | 12.57 |
| 3 | 0.00 | 13.85 | 4.57 | 18.42 |
| 4 | 0.00 | 17.20 | 11.38 | 28.58 |
| 5 | 0.00 | 21.08 | 11.31 | 32.39 |
| 6 | 0.00 | 25.51 | 11.29 | 36.80 |
| 7 | 0.00 | 32.59 | 9.83 | 42.42 |
| 8 | 0.00 | 24.10 | 10.01 | 34.11 |
| 9 | 0.00 | 39.26 | 9.94 | 49.20 |

Lysine was produced in all flasks in comparable amounts in an order of magnitude of approximately 30 to 40 g/l, corresponding to the yield obtained in a standard fermentation using glucose nutrient solution.

### c) Preparation of Dry Powders

### c.1) Spray Drying

250 g of a lysine-comprising liquor with a solids content of approximately 20% by weight (obtained from a maize meal suspension as described in Example 1a and 1b) were introduced at room temperature into a glass beaker and conveyed into a cocurrently operated dual-substance nozzle of a spray tower (Niro, Minor High Tec) by means of a roller pump (type: ISM444, Ismatec). The spray pressure was 4 bar. During the spray process, approximately 2 to 3 g of Sipernat S22 were metered in small portions. The inlet temperature was from 95° C. to 100° C. The pump capacity was adjusted so that the temperature of the product was essentially not below 50° C.

While carrying out the spray drying process, the walls of the spray tower were coated moderately with lysine. The dry powder obtained is visually fine and has good flowability. 23 g of dry powder were obtained.

### c.2) Extrusion

400 g of a lysine-comprising liquor with a solids content of approximately 20% by weight (obtained from a maize meal suspension analogously to Example 1a and 1b) which had been heated for 60 minutes at 80° C. was treated with a PVA solution prepared by dissolving 14 g of polyvinyl alcohol (PVA; M_{w}=10 000 to 190 000 g/mol) in 75 g of water. The pH of the resulting suspension was approximately 7. This suspension was added to approximately 950 g of maize starch (from Roquette) which had initially been placed into a Lödige mixer and mixed at approximately 100-350 rpm.

The mealy, moist, pasty product which was discharged from the mixer and which had a temperature of approximately 30° C. was subsequently fed to a DOME extruder (Fuji Paudal Co. Ltd.) and extruded by a temperature of below 30° C. The extrudate was dried for 120 minutes in a fluidized-bed dryer from BÜCHI at a product temperature of less than 60° C. This gave 600 g of granules.

### c.3) Agglomeration in the Fluidized Bed

500 g of Na₂SO₄ were initially introduced into the cone of a fluidized-bed apparatus Aeromatic MP-1 (Niro Aeromatic; perforation area of the perforated bottom: 12% (12% FF)) and warmed to a temperature of 50° C. 998 g of a lysine-comprising liquor with a solids content of approximately 20% by weight (obtained from a maize meal suspension analogously to Example 1a and 1b) were fed to a dual-substance nozzle (d=1.2 mm) by means of a roller pump and sprayed via this nozzle in top spray position (i.e. from above) onto the solid which had been introduced into the cone. The spray pressure was 1.5 bar. The spray process was interrupted after the addition of 278 g and the addition of a further 320 g of the lysine-comprising liquor (corresponding to a portion of 10 and 20% by weight, respectively, sprayed-on fermentation solid, based on the total solid in the fluidized-bed apparatus) in each case for intermediate drying and sampling (in each case 50 g). The inlet air was adjusted to an amount of in the range of approximately 45 to 60 m³/h and reduced during the drying steps. The inlet air temperature was in the range of from approximately 46° C. to 80° C., during the final drying step in some cases lower. The pump capacity was adjusted so that the temperature of the product was approximately 50° C. and essentially not below 45° C. After cooling, 513 g of product were discharged. The size of the agglomerates of all three product samples taken was in the range of a few hundred micrometers.

### c.4) Contact Drying

240 g of a lysine-comprising liquor with a solids content of approximately 20% by weight (obtained from a corn mill suspension analogously to Example 1a and 1b) were introduced into a 500-ml round-bottomed flask and subsequently concentrated on a rotary evaporator at slightly reduced pressure (880 to 920 mbar). The bath temperature was 140-145° C. After approximately 40 min, the coating produced on the wall of the flask was mechanically comminuted, drying was continued and, after a further 40 min, another comminution step was performed. Drying was subsequently continued and occasionally interrupted in order to perform a further comminution of the residue. The total drying time was 2.5 h. The granules obtained are dark brown and readily flowable. The residual moisture of the granules was 3%. Only small amounts of granules adhered to the wall of the flask.

### Example 2

Using a maize meal hydrolyzate obtained in accordance with Example II.1, a fermentation is carried out analogously to Example 1b), using the strain ATCC13032 lysC^{fbr} which is described in WO 05/059144. The cells are incubated for 48 hours at 30° C. on sterile CM agar (composition table 4; 20 minutes at 121° C.). The cells are subsequently scraped from the plates and resuspended in saline. 25 ml of medium 1 or 2 (see Table 5) in 250 ml Erlenmeyer flasks are in each case inoculated with such an amount of the cell suspension thus prepared that the optical density reaches an OD₆₁₀ value of 1 at 610 nm. The samples are then incubated for 48 hours in a humidified shaker (relative atmospheric humidity 85%) at 200 rpm and 30° C. The lysine concentration in the media is determined by means of HPLC. In all cases, approximately identical amounts of lysine were produced.

The resulting lysine-comprising fermentation liquors were processed as described in Example 1c.2) to give an extrudate.

### Example 3

A maize meal hydrolyzate obtained in accordance with Example II.3a was employed in shake flask experiments using Corynebacterium glutamicum (ATCC13032 lysC^{fbr}) (flasks 1+2). In addition, a wheat meal hydrolyzate (flasks 3+4) and a rye meal hydrolyzate (flasks 5+6) prepared analogously to Example II.3 were used in parallel.

### 3.1) Preparation of the Inoculum

The cells are streaked onto sterile CM+CaAc agar (composition: see Table 7; 20 minutes at 121° C.) and then incubated for 48 hours at 30° C., then inoculated onto a fresh plate and incubated overnight at 30° C. The cells are subsequently scraped from the plates and resuspended in saline. 23 ml of the medium (see Table 8) in 250 ml Erlenmeyer flasks with two baffles are inoculated in each case with such an amount of the cell suspension thus prepared that the optical density reaches an OD₆₁₀ value of 0.5 at 610 nm.

TABLE 7
Composition of the CM + CaAc agar plates

| Concentration | Constituent |
|---|---|
| 10.0 g/l | D-glucose |
| 2.5 g/l | NaCl |
| 2.0 g/l | Urea |
| 5.0 g/l | Bacto peptone (Difco) |
| 5.0 g/l | Yeast extract (Difco) |
| 5.0 g/l | Beef extract (Difco) |
| 20.0 g/l | Casamino acids |
| 20.0 g/l | Agar |

### 3.2) Preparation of Fermentation Liquor

The compositions of the flask media 1 to 6 are listed in Table 8.

In the control medium, a corresponding amount of glucose solution was used instead of meal hydrolyzate.

TABLE 8
Flask media

| | | Flask No | | |
|---|---|---|---|---|
| | | 1 + 2 | 3 + 4 | 5 + 6 |
| Maize | 344 g/kg** | 174 g/l*** | | |
| Wheat | 343 g/kg** | | 175 g/l*** | |
| Rye | 310 g/kg** | | | 194 g/l*** |

| | | |
|---|---|---|
| (NH₄)₂SO₄ | 20 | g/l |
| Urea | 5 | g/l |
| KH₂PO₄ | 0.113 | g/l |
| K₂HPO₄ | 0.138 | g/l |
| ACES | 52 | g/l |
| MOPS | 21 | g/l |
| Citric acid × H₂O | 0.49 | g/l |
| 3,4-Dihydroxybenzoic acid | 3.08 | mg/l |
| NaCl | 2.5 | g/l |
| KCl | 1 | g/l |
| MgSO₄ × 7H₂O | 0.3 | g/l |
| FeSO₄ × 7H₂O | 25 | mg/l |
| MnSO₄ × 4-6H₂O | 5 | mg/l |
| ZnCl₂ | 10 | mg/l |
| CaCl₂ | 20 | mg/l |
| H₃BO₃ | 150 | μg/l |
| CoCl₂ × 6H₂O | 100 | μg/l |
| CuCl₂ × 2H₂O | 100 | μg/l |
| NiSO₄ × 6H₂O | 100 | μg/l |
| Na₂MoO₄ × 2H₂O | 25 | μg/l |
| Biotin (Vit. H) | 1050 | μg/l |
| Thiamine × HCl (Vit B₁) | 2100 | μg/l |
| Nicotinamide | 2.5 | mg/l |
| Pantothenic acid | 125 | mg/l |
| Cyanocobalamin (Vit B₁₂) | 1 | μg/l |
| 4-Aminobenzoic acid (PABA; | 600 | μg/l |
| Vit. H₁) | | |
| Folic acid | 1.1 | μg/l |
| Pyridoxin (Vit. B₆) | 30 | μg/l |
| Riboflavin (Vit. B₂) | 90 | μg/l |
| CSL | 40 | ml/l |

| | |
|---|---|
| pH* | 6.85 |

*to be adjusted with dilute aqueous NaOH solution
**glucose concentration in the hydrolyzate
***amount of weighed-in hydrolyzate per liter of medium

After the inoculation, the flasks were incubated for 48 hours at 30° C. and with shaking (200 rpm) in a humidified shaker. After the fermentation was terminated, the glucose and lysine content was determined by HPLC. The HPLC analyses were carried out using 1100 Series LC Systems from Agilent. Determination of the amino acid formed requires pre-column derivatization with ortho-phthalaldehyde, the product mixture is separated using a Zorbax Extend C18 column from Agilent. The results are compiled in Table 9.

TABLE 9

| Flask No. | Glucose [g/l] | Lysine [g/l] |
|---|---|---|
| 1 | 1.2 | 12.0 |
| 2 | 1.2 | 10.8 |
| 3 | 0.2 | 10.6 |
| 4 | 0.2 | 10.0 |
| 5 | 0.0 | 11.1 |
| 6 | 0.0 | 9.5 |

Lysine was produced in all flasks in comparable amounts in an order of magnitude of approximately 10 to 12 g/l, corresponding to the yield obtained in a standard fermentation using glucose nutrient solution.

The resulting lysine-containing fermentation liquors were processed in accordance with Example 1c.1) to give a flowable powder.

### Example 4

A maize meal hydrolyzate obtained in accordance with Example II.3a was used in shake flask experiments (flasks 1-3). The pantothenate-producing strain was Bacillus PA824 (detailed description in WO 02/061108). In addition, a wheat meal hydrolyzate (flasks 4-6) and a rye meal hydrolyzate (flasks 7-9) prepared analogously to Example II.3 were used in parallel.

### 4.1) Preparation of the Inoculum

42 ml of the preculture medium (see table 10) in 250 ml Erlenmeyer flasks equipped with two baffles are inoculated with in each case 0.4 ml of a frozen culture and incubated for 24 hours at 43° C. with shaking (250 rpm) in a humidified shaker.

TABLE 10
Composition of the preculture medium

| Constituent | Concentration | |
|---|---|---|
| Maltose | 28.6 | g/l |
| Soya meal | 19.0 | g/l |
| (NH₄)₂SO₄ | 7.6 | g/l |
| Monosodium glutamate | 4.8 | g/l |
| Sodium citrate | 0.95 | g/l |
| FeSO₄ × 7H₂O | 9.5 | mg/l |
| MnCl₂ × 4H₂O | 1.9 | mg/l |
| ZnSO₄ × 7H₂O | 1.4 | mg/l |
| CoCl₂ × 6H₂O | 1.9 | mg/l |
| CuSO₄ × 5H₂O | 0.2 | mg/l |
| Na₂MoO₄ × 2H₂O | 0.7 | mg/l |
| K₂HPO₄ × 3H₂O | 15.2 | g/l |
| KH₂PO₄ | 3.9 | g/l |
| MgCl₂ × 6H₂O | 0.9 | g/l |
| CaCl₂ × 2H₂O | 0.09 | g/l |
| MOPS | 59.8 | g/l |

| | |
|---|---|
| pH* | 7.2 |

*to be adjusted with dilute aqueous KOH solution

42 ml of the main culture medium (see Table 11) in 250 ml Erlenmeyer flasks equipped with two baffles are in each case inoculated with 1 ml of preculture.

### 4.2) Preparation of the Fermentation Liquor

The compositions of the flask media 1 to 9 are listed in Table 11.

In the control medium, a corresponding amount of glucose solution was used instead of meal hydrolyzate.

TABLE 11
Flask media

| | | Flask No. | | |
|---|---|---|---|---|
| | | 1-3 | 4-6 | 7-9 |
| Maize | 381.4 g/kg** | 75 g/l*** | | |
| Wheat | 342.0 g/kg** | | 84 g/l*** | |
| Rye | 303.0 g/kg** | | | 94 g/l*** |

| | | |
|---|---|---|
| Soya meal | 19.0 | g/l |
| (NH₄)₂SO₄ | 7.6 | g/l |
| Monosodium glutamate | 4.8 | g/l |
| Sodium citrate | 0.95 | g/l |
| FeSO₄ × 7H₂O | 9.5 | mg/l |
| MnCl₂ × 4H₂O | 1.9 | mg/l |
| ZnSO₄ × 7H₂O | 1.4 | mg/l |
| CoCl₂ × 6H₂O | 1.9 | mg/l |
| CuSO₄ × 5H₂O | 0.2 | mg/l |
| Na₂MoO₄ × 2H₂O | 0.7 | mg/l |
| K₂HPO₄ × 3H₂O | 15.2 | g/l |
| KH₂PO₄ | 3.9 | g/l |
| MgCl₂ × 6H₂O | 0.9 | g/l |
| CaCl₂ × 2H₂O | 0.09 | g/l |
| MOPS | 59.8 | g/l |

| | |
|---|---|
| pH* | 7.2 |

*to be adjusted with dilute aqueous NaOH solution
**glucose concentration in the hydrolyzate
***amount of hydrolyzate weighed in per liter of medium

After the inoculation, the flasks were incubated for 24 hours at 43° C. and with shaking (250 rpm) in a humidified shaker. After the fermentation was terminated, the glucose and pantothenic acid contents were determined by HPLC. The glucose determination was carried out with the aid of an Aminex HPX-87H column from Bio-Rad. The pantothenic acid concentration was determined by means of separation on an Aqua C18 column from Phenomenex. The results are compiled in Table 12.

TABLE 12

| Flask No. | Glucose [g/l] | Pantothenic acid [g/l] |
|---|---|---|
| 1 | 0.00 | 1.75 |
| 2 | 0.00 | 1.70 |
| 3 | 0.00 | 1.73 |
| 4 | 0.10 | 1.80 |
| 5 | 0.10 | 1.90 |
| 6 | 0.19 | 1.96 |
| 7 | 0.12 | 2.01 |
| 8 | 0.12 | 2.12 |
| 9 | 0.13 | 1.80 |

In all flasks, pantothenic acid was produced in comparable amounts in an order of magnitude of approximately from 1.5 to 2 g/l, which is in accordance with the yield achieved in a standard fermentation with glucose nutrient solution.

The resultant pantothenic-acid-comprising fermentation liquors were in some cases processed in accordance with Example 1c.3) to give an agglomerate or in accordance with Example 1c.4) further processed to give a dry, coarse powder.

### Example 5

A maize meal hydrolyzate obtained in accordance with Example II.3a was employed in shake flask experiments using Aspergillus niger (flasks 1-3). In addition, a wheat meal hydrolyzate (flasks 4-6) and a rye meal hydrolyzate (flasks 7-9) prepared analogously to Example II.3 were used in parallel.

### 5.1) Strains

An Aspergillus niger phytase production strain with 6 copies of the phyA gene from Aspergillus ficuum under the control of the glaA promoter was generated analogously to the production of NP505-7, which is described in detail in WO 98/46772. The control used was a strain with 3 modified glaA amplicons (analogously to ISO 505), but without integrated phyA expression cassettes.

### 5.2) Preparation of the Inoculum

20 ml of the preculture medium (see Table 13) in 100 ml Erlenmeyer flasks equipped with a baffle are inoculated with in each case 100 μl of a frozen culture and incubated for 24 hours at 34° C. with shaking (170 rpm) in a humidified shaker.

TABLE 13
Composition of the preculture medium

| Constituent | Concentration | |
|---|---|---|
| Glucose | 30.0 | g/l |
| Peptone from caseine | 10.0 | g/l |
| Yeast Extract | 5.0 | g/l |
| KH₂PO₄ | 1.0 | g/l |
| MgSO₄ × 7H₂O | 0.5 | g/l |
| ZnCl₂ | 30 | mg/l |
| CaCl₂ | 20 | mg/l |
| MnSO₄ × 1H₂O | 9 | mg/l |
| FeSO₄ × 7H₂O | 3 | mg/l |
| Tween 80 | 3.0 | g/l |
| Penicillin | 50000 | IU/l |
| Streptomycin | 50 | mg/l |

| | |
|---|---|
| pH* | 5.5 |

*to be adjusted with dilute sulfuric acid

50 ml of the main culture medium (see Table 14) in 250 ml Erlenmeyer flasks equipped with one baffle are inoculated with in each case 5 ml of preculture.

### 5.3) Preparation of the Fermentation Liquor

The compositions of the flask media 1 to 9 are listed in Table 14.

In the control medium, a corresponding amount of glucose solution was used instead of meal hydrolyzate.

TABLE 14
Flask media

| | | Flask No. | | |
|---|---|---|---|---|
| | | 1-3 | 4-6 | 7-9 |
| Maize | 381.4 g/kg** | 184 g/l*** | | |
| Wheat | 342.0 g/kg** | | 205 g/l*** | |
| Rye | 303.0 g/kg** | | | 231 g/l*** |

| | | |
|---|---|---|
| Peptone from caseine | 25.0 | g/l |
| Yeast Extract | 12.5 | g/l |
| KH₂PO₄ | 1.0 | g/l |
| K₂SO₄ | 2.0 | g/l |
| MgSO₄ × 7H₂O | 0.5 | g/l |
| ZnCl₂ | 30 | mg/l |
| CaCl₂ | 20 | mg/l |
| MnSO₄ × 1H₂O | 9 | mg/l |
| FeSO₄ × 7H₂O | 3 | mg/l |
| Penicillin | 50000 | IU/l |
| Streptomycin | 50 | mg/l |

| | |
|---|---|
| pH* | 5.6 |

*to be adjusted with dilute sulfuric acid
**glucose concentration in the hydrolyzate
***amount of hydrolyzate weighed in per liter of medium

After the inoculation, the flasks were incubated for 6 days at 34° C. and with shaking (170 rpm) in a humidified shaker. After the fermentation was terminated, the phytase activity was determined with the aid of an assay. After the fermentation was terminated, the phytase activity was determined with phytic acid as the substrate and at a suitable phytase activity level (standard: 0.6 U/ml) in 250 mM acetic acid/sodium acetate/Tween 20 (0.1% by weight), pH 5.5 buffer. The assay was standardized for the use in microtiter plates (MTP). 10 μl of the enzyme solution were mixed with 140 μl of 6.49 mM phytate solution in 250 mM sodium acetate buffer, pH 5.5 (phytate: dodecasodium salt of phytic acid). After incubation for 1 hour at 37° C., the reaction was quenched by addition of an equal volume (150 μl) of trichloroacetic acid. One aliquot of this mixture (20 μl) was transferred into 280 μl of a solution comprising 0.32 N H₂SO₄, 0.27% by weight of ammonium molybdate and 1.08% by weight of ascorbic acid. This was followed by incubation for 25 minutes at 50° C. The absorption of the blue solution was measured at 820 nm. The results are compiled in Table 15.

TABLE 15

| | Phytase activity [FTU/ml]* |
|---|---|
| Maize | 433 |
| Wheat | 476 |
| Rye | 564 |
| Control | 393 |

*FTU = Formazine turbidity unit

The resulting phytase-comprising fermentation liquors were processed in accordance with Example 1c.1) to give a powder and in accordance with Example 1c.3) to give a particulate agglomerate.

### Example 6

A maize meal hydrolyzate obtained in accordance with Example II.3a was employed in shake flask experiments using Ashbya gossypii (flasks 1-4). In addition, a wheat meal hydrolyzate (flasks 5-8) and a rye meal hydrolyzate (flasks 9-12) prepared analogously to Example II.3 were used in parallel.

### 6.1) Strain

The riboflavin-producing strain employed is an Ashbya gossypii ATCC 10895 (s.a. Schmidt G, et al. Inhibition of purified isocitrate lyase identified itaconate and oxalate as potential antimetabolites for the riboflavin overproducer Ashbya gossypii. Microbiology 142: 411-417, 1996).

### 6.2) Preparation of the Inoculum

The cells are streaked onto sterile YMG agar (composition: see Table 16; 20 minutes at 121° C.) and then incubated for 72 hours at 28° C.

TABLE 16
Composition of the YMG agar plates

| Constituent | Concentration |
|---|---|
| D-Glucose | 4.0 g/l |
| Yeast extract | 4.0 g/l |
| Malt extract | 10.0 g/l |
| Agar | 30.0 g/l |
| pH | 7.2 |

Thereafter, 50 ml of the preculture medium (see Table 17) in 250 ml Erlenmeyer flasks equipped with two baffles are inoculated in each case with one loop full of cells and incubated for 24 hours at 28° C. with shaking (180 rpm) in a humidified shaker.

TABLE 17
Composition of the preculture medium

| Constituent | Concentration |
|---|---|
| Bacto peptone | 10.0 g/l |
| Yeast extract | 1.0 g/l |
| myo-Inositol | 0.3 g/l |
| D-Glucose | 10.0 g/l |
| pH* | 7.0 |

*to be adjusted with dilute aqueous NaOH solution

50 ml of the main culture medium (see Table 18) in 250 ml Erlenmeyer flasks equipped with two baffles are inoculated with in each case 5 ml of preculture.

### 6.3) Preparation of the Fermentation Liquor

The compositions of the flask media 1 to 12 are detailed in Table 18.

In the control medium, a corresponding amount of glucose solution was used instead of meal hydrolyzate.

TABLE 18
Flask media

| | Flask No. | | |
|---|---|---|---|
| | 1-4 | 5-8 | 9-12 |
| Maize 381.4 g/kg** | 26.2 g/l*** | | |
| Wheat 342.0 g/kg** | | 29.2 g/l*** | |
| Rye 303.0 g/kg** | | | 33.0 g/l*** |

| | |
|---|---|
| Bacto peptone | 10.0 g/l |
| Yeast extract | 1.0 g/l |
| myo-Inositol | 0.3 g/l |
| pH* | 7.0 |

*to be adjusted with aqueous NaOH solution
**glucose concentration in the hydrolyzate
***amount of hydrolyzate weighed in per liter of medium

After the inoculation, the flasks were incubated for 6 days at 28° C. and with shaking (180 rpm) in a humidified shaker. After the fermentation was terminated, the vitamin B₂ content was determined by HPLC. The results are compiled in Table 19.

TABLE 19

| | Vitamin B₂ |
|---|---|
| Maize | 2.73 g/l |
| Wheat | 2.15 g/l |
| Rye | 2.71 g/l |
| Control | 0.12 g/l |

The resulting vitamin-B₂-comprising fermentation liquors were processed in accordance with Example 1c.1) to give a powder and in accordance with Example 1c.3) to give a particulate agglomerate.

### Example 7

A maize meal hydrolyzate obtained in accordance with Example II.3a was employed in shake flask experiments using Corynebacterium glutamicum (flasks 1-3). In addition, a wheat meal hydrolyzate (flasks 4-6) and a rye meal hydrolyzate (flasks 7-9) prepared analogously to Example II.3 were used in parallel.

### 7.1) Strains

Corynebacterium strains which produce methionine are known to the skilled worker. The production of such strains is described for example in Kumar D. Gomes J. Biotechnology Advances, 23 (1):41-61, 2005; Kumar D. et al., Process Biochemistry, 38:1165-1171, 2003; WO 04/024933 and WO 02/18613.

### 7.2) Preparation of the Inoculum

The cells are streaked onto sterile CM+Kan agar (composition: see Table 20; 20 minutes at 121° C.) and then incubated for 24 hours at 30° C. Thereafter, the cells are scraped from the plates and resuspended in saline. 25 ml of the medium (see Table 5) in 250 ml Erlenmeyer flasks equipped with two baffles are inoculated in each case with such an amount of the resulting cell suspension that the optical density reaches an OD₆₁₀ value of 0.5 at 610 nm.

TABLE 20
Composition of the CM + Kan agar plates

| Concentration | | Constituent |
|---|---|---|
| 10.0 | g/l | D-Glucose |
| 2.5 | g/l | NaCl |
| 2.0 | g/l | Urea |
| 10.0 | g/l | Bacto peptone (Difco) |
| 5.0 | g/l | Yeast extract (Difco) |
| 5.0 | g/l | Beef extract (Difco) |
| 20 | μg/ml | Kanamycin |
| 25.0 | g/l | Agar |

### 7.3) Preparation of the Fermentation Liquor

The compositions of the flask media 1 to 9 are listed in Table 21. In the control medium, a corresponding amount of glucose solution was employed instead of meal hydrolyzate.

TABLE 21
Flask media

| Flask No. | | |
|---|---|---|
| 1-3 | 4-6 | 7-9 |

| | | | |
|---|---|---|---|
| Maize 381.4 g/kg** | 157.2 g/l*** | | |
| Wheat 342.0 g/kg** | | 175.6 g/l*** | |
| Rye 303.0 g/kg** | | | 198.0 g/l*** |

| | | |
|---|---|---|
| (NH₄)₂SO₄ | 20 | g/l |
| Urea | 5 | g/l |
| KH₂PO₄ | 0.113 | g/l |
| K₂HPO₄ | 0.138 | g/l |
| ACES | 52 | g/l |
| MOPS | 21 | g/l |
| Citric acid × H₂O | 0.49 | g/l |
| 3,4-Dihydroxybenzoic acid | 3.08 | mg/l |
| NaCl | 2.5 | g/l |
| KCl | 1 | g/l |
| MgSO₄ × 7H₂O | 0.3 | g/l |
| FeSO₄ × 7H₂O | 25 | mg/l |
| MnSO₄ × 4-6H₂O | 5 | mg/l |
| ZnCl₂ | 10 | mg/l |
| CaCl₂ | 20 | mg/l |
| H₃BO₃ | 150 | μg/l |
| CoCl₂ × 6H₂O | 100 | μg/l |
| CuCl₂ × 2H₂O | 100 | μg/l |
| NiSO₄ × 6H₂O | 100 | μg/l |
| Na₂MoO₄ × 2H₂O | 25 | μg/l |
| Biotin (Vit. H) | 1050 | μg/l |
| Thiamine × HCl (Vit B₁) | 2100 | μg/l |
| Nicotinamide | 2.5 | mg/l |
| Pantothenic acid | 125 | mg/l |
| Cyanocobalamin (Vit B₁₂) | 1 | μg/l |
| 4-Aminobenzoic acid (PABA; | 600 | μg/l |
| Vit. H₁) | | |
| Folic acid | 1.1 | μg/l |
| Pyridoxin (Vit. B₆) | 30 | μg/l |
| Riboflavin (Vit. B₂) | 90 | μg/l |
| CSL | 40 | ml/l |
| Kanamycin | 25 | μg/ml |

| | |
|---|---|
| pH* | 6.85 |

*to be adjusted with dilute aqueous NaOH solution
**glucose concentration in the hydrolyzate
***amount of weighed-in hydrolyzate per liter of medium

After the inoculation, the flasks were incubated at 30° C. and with shaking (200 rpm) in a humidified shaker until all of the glucose had been consumed. After the fermentation was terminated, the methioninee content was determined by HPLC (column: Agilent ZORBAX Eclipse AAA; Method according to Eclipse AAA protocol, Technical Note 5980-1193). The results are compiled in Table 22.

TABLE 22

| | Flask | Methioninee [μmol/L] |
|---|---|---|
| Maize | 1 | 9643.1 |
| | 2 | 9509.2 |
| | 3 | 9395.3 |
| Wheat | 4 | 6839.9 |
| | 5 | 7133.9 |
| | 6 | 7028.9 |
| Rye | 7 | 7894.7 |
| | 8 | 7526.5 |
| | 9 | 6998.9 |
| Control | 10 | 1920.8 |
| | 11 | 1916.3 |

The resulting methionine-comprising fermentation liquors were processed as described in Example 1c.4) to give a coarse powder.

### Example 8

A maize meal hydrolyzate obtained in accordance with Example II.3a was employed in shake flask experiments using Bacterium 130Z.

### 8.1) Strain

The succinate-producing strain employed was Bacterium 130Z (ATCC No. 55618).

### 8.2) Preparation of the Fermentation Liquor

50 ml of the main culture medium (see Table 23) in 120 ml serum flasks are inoculated with in each case 1 ml of a frozen culture. Before the serum flasks are sealed, CO₂ is injected in (0.7 bar).

The composition of the medium is listed in Table 23 (cf. U.S. Pat. No. 5,504,004). In the control medium, a corresponding amount of glucose solution was used instead of meal hydrolyzate (final glucose concentration: 100 g/l).

TABLE 23
Medium*

| Constituent | Concentration | |
|---|---|---|
| Maize 381.4 g/kg** | 262 | g/l*** |
| NaCl | 0.1 | g/l |
| K₂HPO₄ | 0.3 | g/l |
| MgCl₂ × 6H₂O | 20 | mg/l |
| CaCl₂ × H₂O | 20 | mg/l |
| (NH₄)₂SO₄ | 0.1 | g/l |
| Biotin | 200 | μg/l |
| CSL | 15.0 | g/l |
| 10% yeast extract | 15.0 | g/l |
| MgCO₃ | 80.0 | g/l |

*treated with gas and dispensed under CO₂/N₂ atmosphere
**glucose concentration in the hydrolyzate
***amount of hydrolyzate weighed in per liter of medium

After the inoculation, the serum flasks were incubated for 46 hours at 37° C. and with shaking (160 rpm) in a shaker. After the fermentation was terminated, the glucose and succinate contents were determined by HPLC. The determination was carried out with the aid of an Aminex HPX-87H column from Bio-Rad. The results are compiled in Table 24.

TABLE 24

| No. | Glucose [g/l] | Succinate [g/l] |
|---|---|---|
| 1 | 30.93 | 42.501 |
| 2 | 29.273 | 44.114 |
| Control | 17.414 | 47.73 |

The resulting succinate-comprising fermentation liquors were processed as described in Example 1c. 1) to give a dry powder.

### Example 9

A maize meal hydrolyzate obtained in accordance with Example II.3a is employed in shake flask experiments using Escherichia coli (flasks 1-3). In addition, a wheat meal hydrolyzate (flasks 4-6) and a rye meal hydrolyzate (flasks 7-9) prepared analogously to Example II.3 are used in parallel.

### 9.1) Strain

Escherichia coli strains which produce L-threonine are known to the skilled worker. The production of such strains is described for example in EP 1013765 A1, EP 1016710 A2, U.S. Pat. No. 5,538,873.

### 9.2) Preparation of the Inoculum

The cells are streaked onto sterile LB agar. If suitable resistance genes exist as markers in the strain in question, antibiotics are added to the LB agar. Substances which can be used for this purpose are, for example, kanamycin (40 μg/ml) or ampicillin (100 mg/l). The strains are incubated for 24 hours at 30° C. After the cells have been streaked onto sterile M9 glucose minimal medium supplemented with methionine (50 μg/ml), kanamycin (40 μg/ml) and homoserin (10 μg/l), they are incubated for 24 hours at 30° C. Thereafter, the cells are scraped from the plates and resuspended in saline. 25 ml of the medium (see Table 25) in 250 ml Erlenmeyer flasks equipped with two baffles are inoculated in each case with such an amount of the cell suspension thus prepared that the optical density reaches an OD₆₁₀ value of 0.5 at 610 nm.

### 9.3) Preparation of the Fermentation Liquor

The compositions of the flask media 1 to 9 are listed in Table 25. In the control medium, a corresponding amount of glucose solution is used instead of meal hydrolyzate.

TABLE 25
Flask media

| | Flask No. | | |
|---|---|---|---|
| | 1-3 | 4-6 | 7-9 |
| Maize 381.4 g/kg** | 157.2 g/l*** | | |
| Wheat 342.0 g/kg** | | 175.6 g/l*** | |
| Rye 303.0 g/kg** | | | 198.0 g/l*** |

| | | |
|---|---|---|
| (NH₄)₂SO₄ | 22 | g/l |
| K₂HPO₄ | 2 | g/l |
| NaCl | 0.8 | g/l |
| MgSO₄ × 7H₂O | 0.8 | g/l |
| FeSO₄ × 7H₂O | 20 | mg/l |
| MnSO₄ × 5H₂O | 20 | mg/l |
| Thiamine × HCl (Vit B₁) | 200 | mg/l |
| Yeast extract | 1.0 | g/l |
| CaCO₃ (sterilized separately) | 30 | g/l |
| Kanamycin | 50 | mg/l |
| Ampicillin | 100 | mg/l |

| | |
|---|---|
| pH* | 6.9 ± 0.2 |

*to be adjusted with dilute aqueous NaOH solution
**glucose concentration in the hydrolyzate
***amount of weighed-in hydrolyzate per liter of medium

After the inoculation, the flasks are incubated at 30° C. and with shaking (200 rpm) in a humidified shaker until all of the glucose has been consumed. After the fermentation has been terminated, the L-threonine content can be determined by reversed-phase HPLC as described by Lindroth et al., Analytical Chemistry 51:1167-1174, 1979.

The resulting threonine-comprising fermentation liquors were further processed in accordance with Examples 1c.1) to 1c.3) to give a powder, an extrudate or an agglomerate.

### Example 10

Using suitable strains, the other L-amino acids glutamate, histidine, proline and arginine are prepared analogously to the procedure of Example 9. The strains in question are described for example in EP 1016710.

The resulting amino-acid-comprising fermentation liquors can be further processed in accordance with Example 1c.1) to 1c.3) to give a dry product.

### Example 11

A partially saccharified maize meal hydrolyzate was employed in shake flask experiments using Aspergillus niger.

### 11.1) Liquefaction and (Partial) Saccharification

The liquefaction was carried out analogously to Example II.3a. After the suspension had been cooled to 61° C. and the pH adjusted to 4.3, 5.38 ml (=1.5% by weight of enzyme/dry matter) of Dextrozyme GA (Novozymes A/S) were added. In each case 10, 15, 20, 30, 45 and 60 minutes after the addition of the enzyme, 50 g of sample were taken and suspended in 25 ml of sterile ice-cold fully demineralized water. The samples were placed into an ice bath and immediately employed in the flask test. No inactivation of the enzyme took place.

### 11.2) Fermentation

The strain used in Example 5.1) was employed. The inoculum was prepared as described in Example 5.2).

To prepare the fermentation liquor, the flask medium compositions listed in Table 29 were used. Two flasks were prepared with each sample.

TABLE 29
Flask media

| | | |
|---|---|---|
| Maize | 10 | g/l*** |
| Peptone from caseine | 25.0 | g/l |
| Yeast Extract | 12.5 | g/l |
| KH₂PO₄ | 1.0 | g/l |
| K₂SO₄ | 2.0 | g/l |
| MgSO₄ × 7H₂O | 0.5 | g/l |
| ZnCl₂ | 30 | mg/l |
| CaCl₂ | 20 | mg/l |
| MnSO₄ × 1H₂O | 9 | mg/l |
| FeSO₄ × 7H₂O | 3 | mg/l |
| Penicillin | 50000 | IU/l |
| Streptomycin | 50 | mg/l |

| | |
|---|---|
| pH* | 5.6 |

*to be adjusted with dilute sulfuric acid
***amount of partially saccharified hydrolyzate weighed in per liter of medium

After the inoculation, the flasks were incubated for 6 days at 34° C. and with shaking (170 rpm) in a humidified shaker. After the fermentation was terminated, the phytase activity was determined with the aid of an assay (as described in Example 5.3). The results are compiled in Table 30.

TABLE 30

| Termination of the standard | | |
|---|---|---|
| saccharification procedure | | Phytase activity |
| after × minutes | Flask | [FTU/ml] |
| 10 | 1 | 425 |
| | 2 | 387 |
| 15 | 3 | 312 |
| | 4 | 369 |
| 20 | 5 | 366 |
| | 6 | 316 |
| 30 | 7 | 343 |
| | 8 | 454 |
| 45 | 9 | 372 |
| | 10 | 358 |
| 60 | 11 | 298 |
| | 12 | 283 |

The resulting phytase-comprising fermentation liquors were processed in accordance with Examples 1c.2) and 1c.3) to give an extrudate or an agglomerate.

### Example 12

A partially saccharified maize meal hydrolyzate was employed in shake flask experiments using Corynebacterium glutamicum.

### 12.1) Liquefaction and (Partial) Saccharification

The liquefaction was carried out analogously to Example II.3a. After the suspension had been cooled to 61° C. and the pH adjusted to 4.3, 5.38 ml (=1.5% by weight of enzyme/dry matter) of Dextrozyme GA (Novozymes A/S) were added. In each case 10, 15, 20, 30, 45 and 60 minutes after the addition of the enzyme, 50 g of sample were taken and suspended in 25 ml of sterile ice-cold fully demineralized water. The samples were placed into an ice bath and immediately employed in the flask test. No inactivation of the enzyme took place.

### 12.2) Fermentation

The strain used in Example 3) was employed. The inoculum was prepared as described in Example 3.1).

To prepare the fermentation liquor, the flask medium compositions listed in Table 31 were used. Two flasks were prepared with each sample.

TABLE 31
Flask media

| | | |
|---|---|---|
| Maize | 4.5 | g/l*** |
| (NH₄)₂SO₄ | 20 | g/l |
| Urea | 5 | g/l |
| KH₂PO₄ | 0.113 | g/l |
| K₂HPO₄ | 0.138 | g/l |
| ACES | 52 | g/l |
| MOPS | 21 | g/l |
| Citric acid × H₂O | 0.49 | g/l |
| 3,4-Dihydroxybenzoic acid | 3.08 | mg/l |
| NaCl | 2.5 | g/l |
| KCl | 1 | g/l |
| MgSO₄ × 7H₂O | 0.3 | g/l |
| FeSO₄ × 7H₂O | 25 | mg/l |
| MnSO₄ × 4-6H₂O | 5 | mg/l |
| ZnCl₂ | 10 | mg/l |
| CaCl₂ | 20 | mg/l |
| H₃BO₃ | 150 | μg/l |
| CoCl₂ × 6H₂O | 100 | μg/l |
| CuCl₂ × 2H₂O | 100 | μg/l |
| NiSO₄ × 6H₂O | 100 | μg/l |
| Na₂MoO₄ × 2H₂O | 25 | μg/l |
| Biotin (Vit. H) | 1050 | μg/l |
| Thiamine × HCl (Vit B₁) | 2100 | μg/l |
| Nicotinamide | 2.5 | mg/l |
| Pantothenic acid | 125 | mg/l |
| Cyanocobalamin (Vit B₁₂) | 1 | μg/l |
| 4-Aminobenzoic acid (PABA; | 600 | μg/l |
| Vit. H₁) | | |
| Folic acid | 1.1 | μg/l |
| Pyridoxin (Vit. B₆) | 30 | μg/l |
| Riboflavin (Vit. B₂) | 90 | μg/l |
| CSL | 40 | ml/l |

| | |
|---|---|
| pH* | 6.85 |

*to be adjusted with dilute aqueous NaOH solution
***amount of partially saccharified hydrolyzate weighed in per liter of medium

After the inoculation, the flasks were incubated for 48 hours at 30° C. and with shaking (200 rpm) in a humidified shaker. After the fermentation was terminated, the glucose and lysine contents were determined by HPLC. The HPLC analyses were carried out with Agilent 1100 Series LC systems. The glucose was determined with the aid of an Aminex HPX-87H column from Bio-Rad. The amino acid concentration was determined by means of high-pressure liquid chromatography on an Agilent 1100 Series LC system HPLC. Pre-column derivatization with ortho-phthalaldehyde permits the quantification of the amino acids formed, the amino acid mixture is separated using a Hypersil AA column (Agilent). The results are compiled in Table 32.

TABLE 32

| Termination of the standard | | |
|---|---|---|
| saccharification process | | |
| after × minutes | Flask | Lysine [g/l] |
| 10 | 1 | 15.05 |
| | 2 | 11.71 |
| | 3 | 14.24 |
| 15 | 4 | 14.91 |
| | 5 | 15.27 |
| | 6 | 12.20 |
| 20 | 7 | 13.19 |
| | 8 | 13.65 |
| | 9 | 11.14 |
| 30 | 10 | 15.38 |
| | 11 | 12.45 |
| | 12 | 11.56 |
| 45 | 13 | 13.13 |
| | 14 | 14.64 |
| | 15 | 13.48 |
| 60 | 16 | 14.58 |
| | 17 | 13.72 |
| | 18 | 14.27 |

The resulting lysine-comprising fermentation liquors were processed in accordance with Examples 1c.1) or 1c.4) to give a powder or a granulate.

1. A process for the production of at least one nonvolatile microbial metabolite in solid form by sugar-based microbial fermentation, in which process a microorganism strain which produces the desired metabolite(s) is grown using a sugar-containing liquid medium with a monosaccharide content of more than 20% by weight based on the total weight of the liquid medium, and the volatile constituents of the fermentation liquor are subsequently largely removed, production of the sugar-containing liquid medium comprising:
a1) production of a millbase by milling a starch feedstock selected from cereal grains; and
a2) liquefying the millbase in an aqueous liquid in the presence of at least one starch-liquefying enzyme, followed by saccharification using at least one saccharifying enzyme,
wherein, for liquefaction purposes, at least a portion of the millbase is added continuously or batchwise to the aqueous liquid in the course of the liquefaction.

2. The method according to claim 1, comprising
a) preparing the sugar-containing liquid medium with a monosaccharide content of more than 20% by weight as described in steps a1) and a2), where the sugar-containing liquid medium also comprises nonstarchy solid constituents of the starch feedstock;
b) utilizing the sugar-containing liquid medium in a fermentation in order to produce the nonvolatile metabolite(s) and
c) obtaining the nonvolatile metabolite(s) in solid form together with at least part of the nonstarchy solid constituents of the starch feedstock from the fermentation liquor by removing at least some of the volatile constituents of the fermentation liquor.

3. The process according to claim 2, wherein the sugar-containing liquid medium prepared in step a) comprises at least 20% by weight of the nonstarchy solid constituents of the starch feedstock.

4. The process according to claim 1, wherein the millbase is liquefied in an aqueous liquid in the presence of at least one α-amylase and subsequently saccharified using at least one glucoamylase.

5. The method according to claim 4, wherein a portion of the at least one α-amylase is added to the aqueous liquid during the liquefaction in step a2).

6. The method according to claim 1, wherein the cereal is selected among maize, rye, triticale and wheat grains.

7. The method according to claim 1, wherein the millbase obtained during grinding in step a1) comprises at least 50% by weight of meal particles with a particle size of more than 100 μm.

8. The method according to claim 1, wherein the liquefaction and saccharification of the millbase in step a2) is carried out in such a way that the viscosity of the liquid medium is not more than 20 Pas.

9. The method according to claim 1, wherein at least 25% by weight of the total amount of the millbase added during the liquefaction are added at the temperature above the gelling temperature of the starch present in the millbase.

10. The method according to claim 1, wherein a sugar-containing liquid medium with a monosaccharide content of more than 30% by weight is prepared.

11. The method according to claim 1, wherein at least one phytase is added to the sugar-containing liquid medium before the fermentation step.

12. The method according to claim 1, wherein the nonvolatile metabolite(s) which has (have) been produced is (are) selected among organic mono-, di- and tricarboxylic acids which optionally have hydroxyl groups attached to them and which have 3 to 10 carbon atoms, among proteinogenic and nonproteinogenic amino acids, purine bases, pyrimidine bases; nucleosides, nucleotides, lipids; saturated and unsaturated fatty acids; diols having 4 to 10 carbon atoms, higher-functionality alcohols having 3 or more hydroxyl groups, longer-chain alcohols having at least 4 carbon atoms, carbohydrates, aromatic compounds, vitamins, provitamins, cofactors, nutraceuticals, proteins, carotenoids, ketones having 3 to 10 carbon atoms, lactones, biopolymers and cyclodextrins.

13. The method according to claim 1, wherein the nonvolatile metabolites prepared are selected among enzymes, amino acids, vitamins, disaccharides, aliphatic mono- and dicarboxylic acids having 3 to 10 carbon atoms, aliphatic hydroxycarboxylic acids having 3 to 10 carbon atoms, ketones having 3 to 10 carbon atoms, alkanols having 4 to 10 carbon atoms and alkanediols having 3 to 10 carbon atoms.

14. The method according to claim 1, wherein the microorganisms are selected among natural or recombinant microorganisms which produce at least one of the following metabolites: enzymes, amino acids, vitamins, disaccharides, aliphatic mono- and dicarboxylic acids having 3 to 10 carbon atoms, aliphatic hydroxycarboxylic acids having 3 to 10 carbon atoms, ketones having 3 to 10 carbon atoms, alkanols having 4 to 10 carbon atoms and alkanediols having 3 to 10 carbon atoms.

15. The method according to claim 14, wherein the microorganisms are selected among the genera Corynebacterium, Bacillus, Ashbya, Escherichia, Aspergillus, Alcaligenes, Actinobacillus, Anaerobiospirillum, Lactobacillus, Propionibacterium, Clostridium and Rhizopus, in particular among strains of Corynebacterium glutamicum, Bacillus subtilis, Ashbya gossypii, Escherichia coli, Aspergillus niger, Alcaligenes latus, Anaerobiospirillum succiniproducens, Actinobacillus succinogenes, Lactobacillus delbrückii, Lactobacillus leichmannii, Propionibacterium arabinosum, Propionibacterium schermanii, Propionibacterium freudenreichii, Clostridium propionicum, Clostridium acetobutlicum, Clostridium formicoaceticum, Rhizopus oryzae and Rhizopus arrhizus.

16. The method according to claim 1, wherein no more than 30% by weight of the solids present in the fermentation liquor are removed before removing the volatile constituents of the fermentation liquor.

17. The method according to claim 1, wherein the liquid phase of the fermentation liquor is removed without previously separating insoluble constituents of the fermentation liquor, and the metabolite is obtained together with all of the insoluble constituents of the fermentation liquor.

18. The method according to claim 1, wherein the at least one nonvolatile metabolite is obtained from the fermentation liquor in solid form together with the totality of all insoluble constituents without previously removing the insoluble constituents of the fermentation liquor.

19. The method according to claim 1, wherein the volatile constituents of the fermentation liquor are removed from the fermentation liquor down to a residual moisture content of from 0.2 to 20% by weight, preferably from 1 to 15% by weight and especially preferably from 5 to 10% by weight, based on the total dry weight of the solid constituents.

20. The method according to claim 1, wherein the fermentation liquor is spray dried, fluidized-bed-dried or freeze-dried to remove the volatile constituents.

21. The method according to claim 20, wherein one or more drying adjuvants are used.

22. A solid formulation of a metabolite, obtained by the method according to claim 1.

23. The formulation according to claim 22, comprising:
A) >10 to 80% by weight of at least one nonvolatile metabolite;
B) 1 to 50% by weight of biomass from the fermentation which produces the nonvolatile metabolite;
C) 1 to 50% by weight of nonstarchy solid constituents of the starch feedstock from the fermentation liquor; and
D) 0 to 400% by weight, based on the total weight of components A, B and C, of conventional formulation adjuvants;
the parts by weight A, B and C totaling 100% by weight.

24. The formulation according to claim 23, comprising at least 5% by weight of dietary fibers, based on the total weight of the formulation.

25. A method for human or animal nutrition comprising utilizing the formulation according to claim 22 for human or animal nutrition.

26. A method for the treatment of textiles leather, cellulose, paper or surfaces comprising utilizing the formulation according to claim 22 for the treatment of textiles, leather, cellulose, paper or surfaces.
